(19) United States
(12) Patent Application Publication
(10) Pub. No.: US 2021/0187073 A1
(43) Pub. Date: Jun. 24, 2021

(54) TREATMENT OF HEPATITIS DELTA VIRUS INFECTION WITH INTERFERON LAMBDA

(71) Applicant: Eiger Biopharmaceuticals, Inc., Palo Alto, CA (US)

(72) Inventors: Ingrid CHOONG, Palo Alto, CA (US); Eduardo Bruno MARTINS, Palo Alto, CA (US)

(21) Appl. No.: 17/268,657

(22) PCT Filed: Aug. 23, 2019
(86) PCT No.: PCT/US2019/048038

§ 371 Date: Feb. 17, 2021

Related U.S. Application Data
(60) Provisional application No. 62/721,763, filed on Aug. 23, 2018.
(60) Provisional application No. 62/823,530, filed on Mar. 25, 2019.
(60) Provisional application No. 62/831,548, filed on Apr. 9, 2019.

(51) Int. Cl. A61K 38/21; A61K 47/60; A61K 31/675; A61K 31/7072; A61K 31/522; A61P 31/14

(52) U.S. Cl. CPC A61K 38/21; A61K 47/60; A61P 31/14; A61K 31/7072; A61K 31/522; A61K 31/675

(57) ABSTRACT

Methods of treating a hepatitis delta virus (HDV) infection in a human subject are provided. In some embodiments, the method comprises subcutaneously administering to the subject a therapeutically effective amount of pegylated interferon lambda-1a for at least 48 weeks.

### CROSS-REFERENCE

This application claims priority to provisional applications No. 62/831,548 (filed Apr. 9, 2019), No. 62/823,530 (filed Mar. 25, 2019), and No. 62/721,763 (filed Aug. 23, 2018), each of which is incorporated herein by reference.

### FIELD OF INVENTION

The present invention provides methods for treating viral hepatitis resulting from hepatitis delta virus (HDV) infection, and so relates to the fields of chemistry, medicinal chemistry, medicine, molecular biology, and pharmacology.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 097854-1152075-002410_PC_SL.TXT, created on Aug. 8, 2019, and having a size of 1.97 MB and is filed concurrently with the specification. The sequence listing contained in this ASCII formatted document is part of the specification and is herein incorporated by reference in its entirety.

### BACKGROUND OF THE INVENTION

Hepatitis delta virus (HDV) causes the most severe form of chronic viral hepatitis. HDV presents as a co-infection with hepatitis B virus (HBV). Chronic HDV and HBV co-infection worsens preexisting HBV-related liver damage and leads to liver cirrhosis, hepatic decompensation, and hepatocellular carcinoma. See, Negro, Cold Spring Harb Perspect Med, 2014, 4:a021550; Höner zu Siederdissen, Visc Med, 2016, 32:86-94; Lau, Hepatology, 1999, 30:546-549. Subjects who are co-infected with both HDV and HBV are more likely to die of complications from liver disease compared to subjects infected with HBV alone. See, Alavian et al., J Res Med Sci, 2012, 17:967-974.

Interferon alpha therapy for the treatment of HDV has been described. In a Hep-Net International Delta Intervention Trial 1 (HIDIT-1) study, published in 2011, it was found that 28% of subjects receiving peginterferon alfa-2a therapy had clearance of HDV RNA 24 weeks after the end of treatment. Wedemeyer et al., N Engl J Med, 2011, 364:322-331. However, a five year long-term follow-up of subjects treated in the HIDIT-1 study found that more than 50% of the subjects exhibited late HDV RNA relapse. Heidrich et al., Hepatology, 2014, 60:87-97.

In contrast to interferon alpha, which mediates its effects by signaling through interferon alpha receptors that are widely expressed by many different cell types, interferon lambda signals through a different class of receptors (the interferon lambda receptors) that have a restricted cellular expression pattern. Interferon lambda also exhibits distinct antiviral activities from interferon alpha, due in part to the differences in expression of the interferon receptors. In a comparative study of pegylated interferon alfa and a pegylated interferon lambda for the treatment of HBV (Chan et al., J. Hepatology, 2016, 64:1011-1019), it was found that although pegylated interferon lambda produced more pronounced declines in viremia as compared to pegylated interferon alfa at the midpoint of treatment (24 weeks), by the end of the treatment period there was no difference between pegylated interferon alfa and pegylated interferon lambda treatment, and post-treatment there was a greater virologic rebound in the pegylated interferon lambda treatment group. HBV/HDV co-infected mice receiving pegylated interferon alfa for four weeks exhibited a 2.2 log reduction in HDV-RNA levels, while mice receiving pegylated interferon lambda for four weeks exhibited a 1.5 log reduction in HDV-RNA levels (Giersch et al., 2013).

To date, the efficacy of long-term pegylated interferon lambda therapy for the treatment of HDV has not been described. There continues to be an ongoing need for agents to treat HDV infection.

### BRIEF SUMMARY OF THE INVENTION

In one aspect, methods of treating a hepatitis delta virus (HDV) infection in a human subject are provided. In some embodiments, the method comprises subcutaneously administering to the subject a therapeutically effective amount of pegylated interferon lambda-1a for at least 48 weeks.

In some embodiments, the pegylated interferon lambda-1a is administered at a dose of 180 micrograms once a week (QW). In some embodiments, the pegylated interferon lambda-1a is administered at a dose of 120 micrograms QW. In some embodiments (i) 160-180 micrograms pegylated interferon lambda-1a is administered per week for a first treatment period and then 150-70 micrograms per week for a second treatment period; or (ii) 180 micrograms per week for a first treatment period and then between 170-120 micrograms per week for a second treatment period, wherein the doses for each of (i) and (ii) may be divided into more than one dose per week.

In some embodiments, the method comprises administering the pegylated interferon lambda-1a at a dose of 180 micrograms QW for a first treatment period and then at a dose of 120 micrograms QW for a second treatment period. In some embodiments, the method comprises administering the pegylated interferon lambda-1a at a dose of 120 micrograms QW for a first treatment period and then at a dose of 80 micrograms QW for a second treatment period. In some embodiments, the first treatment period is longer than the second treatment period. In some embodiments, the second treatment period is longer than the first treatment period. In some embodiments, the first treatment period and the second treatment period are the same length of time. In some embodiments, the first treatment period has a duration of at least 8 weeks. In some embodiments, the first treatment period has a duration of 8-12 weeks. In some embodiments, the method further comprises administering the pegylated interferon lambda-1a at a dose of 80 micrograms QW for a third treatment period. In some embodiments, the method comprises administering the pegylated interferon lambda-1a at a dose of 180 micrograms QW for a first treatment period and then at a dose of 120 micrograms QW for a second treatment period followed by administering a dose of 60 micrograms-110 micrograms QW for a third treatment period.

In some embodiments, the method comprises administering the pegylated interferon lambda-1a at a first dose of 180 micrograms QW for a first treatment period, at a second dose of 120 micrograms QW for a second treatment period, and at a third dose of 110-80 micrograms QW for a third treatment period. In some embodiments, the first treatment period has a duration of at least 8 weeks. In some embodiments, the first treatment period has a duration of 8-12 weeks or 1-8 weeks or 2-12 weeks.

In some embodiments, treatment results in a reduction of HDV viral load in the subject of at least 2.0 log HDV RNA IU/mL serum. In some embodiments, treatment results in an HDV viral load that is below the level of detection. In some embodiments, prior to the onset of treatment, the subject has a serum alanine aminotransferase (ALT) level that is above the upper limit of normal (ULN), and the course of treatment results in an improvement in serum ALT level in the subject to a level that is within the ULN.

In some embodiments, prior to treatment, the subject has a baseline viral load of up to about 10⁴ HDV RNA copies per mL serum or plasma.

In some embodiments, subjects having a low viral load have a higher percentage of BLQ response at 48 weeks and at 24 weeks post treatment.

In one embodiment, the Lambda 180 μg treatment group, response rates differed between subjects with high (>4 logs) versus low (≤4 logs) baseline viral load. In one embodiment, at week 48, 38-43% and 33-40% of subjects with high versus low baseline viral loads respectively, reached HDV RNA levels BLQ. In another embodiment, at week 72, the difference between these two groups became more prominent, with 50-60% of subjects in the low baseline viral load reaching BLQ versus 25-29% in the high baseline viral load meeting this endpoint.

In one embodiment, at week 48, 25-29% and 33-40% of subjects with high versus low baseline viral loads respectively, reached undetectable levels of HDV RNA. In one embodiment, at week 72, there were difference between these two groups were consistent with the 48 week measure, with 33-40% of subjects in the low baseline viral load reaching BLQ versus 25-29% in the high baseline viral load meeting this endpoint.

In some embodiments, the method further comprises administering to the subject a nucleoside or nucleotide analog. In some embodiments, the nucleoside or nucleotide analog is lamuvidine, adefovir, telbivudine, entecavir, or tenofovir.

In some embodiments, the subject has compensated liver disease with or without cirrhosis. In some embodiments, the subject has compensated liver disease with cirrhosis.

Other aspects and embodiments are disclosed infra.

### BRIEF DESCRIPTION OF THE DRAWINGS

FIG. 1. Interferon lambda demonstrates rapid decline in HDV RNA at week 24. Mean change in log HDV RNA is shown through week 24.

FIG. 2. Interferon lambda demonstrates rapid decline in HDV RNA at week 48. Mean change in log HDV RNA is shown through week 48. Inset table shows number and percentage of subjects having ≥2 log₁₀ decline in HDV RNA and number and percentage of subjects having an HDV RNA level below the limit of quantitation at weeks 4, 8, 12, 24, and 48.

FIG. 3. Interferon lambda at 180 mcg results in higher response rates as compared to interferon lambda at 120 mcg. Graph shows mean HDV RNA decline through week 48 for subjects treated with 180 mcg or 120 mcg interferon lambda. Inset table shows mean log decline in HDV RNA, number and percentage of subjects having ≥2 log₁₀ decline in HDV RNA, and number and percentage of subjects having an HDV RNA level below the limit of quantitation, at the 120 mcg and 180 mcg doses.

FIG. 4. Demonstrates the durability of the virologic response.

FIG. 5. Demonstrates ALT normalization with Interferon lambda.

FIG. 6. Demonstrates in conjunction with Table 5 that the response to Interferon lambda is durable.

FIG. 7. Demonstrates HBsAg reduction with Interferon lambda.

FIG. 8. Time course of HDV RNA of Responders at Week 48 of treatment.

FIG. 9. Further demonstrates the durability of virologic response.

FIG. 10. Demonstrates a composite endpoint of ALT normalization and 2 log₁₀ decline or BLQ.

FIG. 11. A comparison of Lambda to PEG INF alpha demonstrating a 0% sustained virologic response (SVR) achieved with PEG INF alpha and a 36% durable virologic response (DVR) achieved with Lambda.

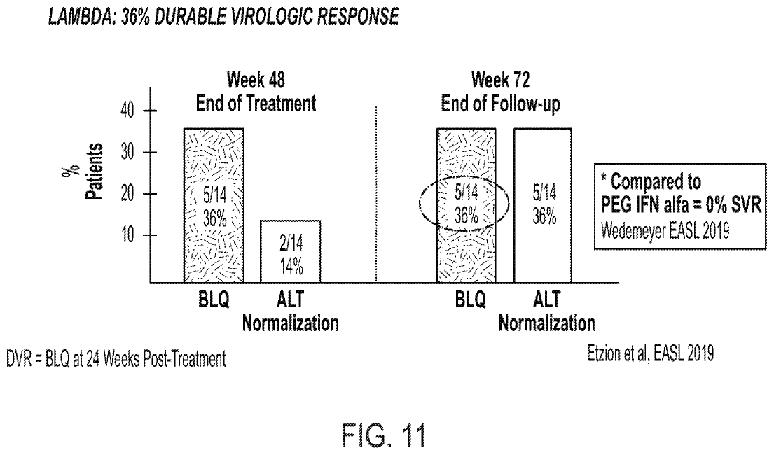

### DETAILED DESCRIPTION OF THE INVENTION

### I. Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.”

The singular forms “a,” “an,” and “the” include plural referents unless the context clearly dictates otherwise. Thus, for example, reference to “a compound” includes a plurality of compounds.

The term “administration” refers to introducing a compound, a composition, or an agent of the present disclosure into a host, such as a human. In the context of the present disclosure, one preferred route of administration of the agents is subcutaneous administration. Other routes of administration include intravenous administration and oral administration.

The term “baseline,” unless otherwise specified or apparent from context, refers to a measurement (of, e.g., viral load, subject condition, ALT level) made prior to a course of therapy.

The term “comprising” is intended to mean that the compounds, compositions and methods include the recited elements, but does not exclude others. “Consisting essentially of” when used to define compounds, compositions and methods, shall mean excluding other elements that would materially affect the basic and novel characteristics of the claimed invention. Embodiments defined by each of these transition terms are within the scope of this invention.

The terms “course of treatment” and “course of therapy” are used interchangeably herein, and refer to the medical interventions made after a subject is diagnosed, e.g., as being infected with HDV and in need of medical intervention. Medical interventions include, without limitation, the administration of drugs for a period of time, typically, for HDV infected subjects, at least one and typically several or many months or even years.

The term “HDV RNA viral load” or “viral load” of a human serum or plasma sample refers to the amount of HDV RNA in a given amount of a human serum or plasma sample. HDV RNA is generally detected by quantitative real-time reverse transcription-polymerase chain reaction (qRT-PCR) assays. In such assays, the amount of signal generated during the assay is proportional to the amount of HDV RNA in the sample. The signal from the test sample is compared to that of a dilution series of a quantified Hepatitis Delta RNA standard, and a copy number of genome copies is calculated. See, e.g., Kodani et al., 2013, J. Virol. Methods, 193(2), 531; Karatayli et al., 2014, J. Clin. Virol, 60(1), 11. HDV RNA viral load may be reported as RNA copies per mL serum (or plasma) or using International Units (IU) per mL serum (or plasma). See, Chudy et al., 2013, Collaborative Study to establish a World Health Organization International standard for hepatitis D virus RNA for nucleic acid amplification technique (NAT)-based assays.” WHO Expert Committee on Biological Standardization WHO/BS/2013.2227. A commercially available assay is available from ARUP Laboratories (Salt Lake City, Utah). The limit of detection for the ARUP HDV RNA assay has been reported to be 31 IU/mL. Analytik Jena AG (Germany) offers the RoboGene® HDV RNA Quantification Kit 2.0, which is CE-IVD certified with WHO standard references to assess the response to antiviral treatment. The limit of detection for the RoboGene® assay is reported to be 6 IU/mL. Reference to a “viral load” without specified units (e.g., “a viral load of less than 100”) refers to copies of HDV RNA per mL serum, unless otherwise indicated or apparent from context. Unless otherwise specified, reference to “below the level of detection” means below 8 IU/mL.

HDV levels are generally presented using log₁₀ units. HDV RNA levels may be presented in units of “RNA copies per mL” or as “International Units (IU) per mL.” See, Chudy et al., 2013, Collaborative Study to establish a World Health Organization International standard for hepatitis D virus RNA for nucleic acid amplification technique (NAT)-based assays.” WHO Expert Committee on Biological Standardization WHO/BS/2013.2227. Both units are used in this specification. As used herein, recitation of “HDV RNA copies per mL,” (when not otherwise specified and not including discussions related to clinical trial results, e.g., as presented in the examples) should be read, for purposes of written description or basis, as referring to “HDV RNA copies/mL or HDV IU/mL.” Where a specific quantity of HDV RNA copies per mL is recited, a multiplier of 1.2 may be applied, for the purposes of written description and support, to convert the quantity of HDV RNA copies/mL to the quantity of IU/mL. For example, “120 HDV RNA copies per mL” should be read as “120 copies/mL or 100 IU/mL.”

Changes in HDV RNA levels may be represented as a “log reduction” following the normal conventions of virology. For example, a 1 log₁₀ reduction (i.e., −1 log₁₀) in viral load (e.g., from 7 log₁₀ to 6 log₁₀) is a 10-fold reduction, and a 2 log₁₀ reduction (i.e., −2 log₁₀) in viral load (e.g., from 7 log₁₀ to 5 log) is a 100-fold reduction. A reduction from 4 log RNA copies/mL to 3 log₁₀ RNA copies/mL is equivalent to a reduction from 4 log₁₀ IU/mL to 3 log₁₀ IU/mL.

The term “HDV infection” with respect to a human (host) refers to the fact that the host is suffering from HDV infection. Typically, an HDV infected human host will have a viral load of HDV RNA of at least about 2 log₁₀ HDV RNA copies/mL of host serum or plasma or 10² copies of HDV-RNA/mL of host serum or plasma, often at least about 3 log₁₀ HDV RNA copies/mL of host serum or plasma or 10³ copies of HDV-RNA/mL of host serum or plasma, and, often, especially for subjects not on any therapy, at least about 4 log₁₀ HDV RNA copies/mL of host serum or plasma or 10⁴ copies of HDV-RNA/mL of host serum or plasma, such as about 4 log₁₀ HDV RNA copies/mL of host serum or plasma to 8 log₁₀ HDV RNA copies/mL of host serum or plasma or 10⁴-10⁸ copies of HDV-RNA/mL of host serum or plasma. As used herein, the term “chronic HDV infection” with respect to a human host refers to an HDV infection that has persisted in the human host for at least 6 months, as documented by a positive HDV antibody (Ab) test and/or detectable HDV RNA by qRT-PCR. Diagnosis and pathogenesis of HDV is described, for example, in Wedemeyer et al., Nat. Rev. Gastroenterol. Hepatol, 2010, 7:31-40.

The term “Lower Limit of Quantification” refers to the lowest concentration of a substance of analyte (e.g., a viral titer) that can be reliably quantified by a particular assay within a stated confidence limit.

The terms “subject,” “host,” or “subject,” are used interchangeably and refer to a human infected with HDV, including subjects previously infected with HDV in whom virus has cleared.

The term “pharmaceutical composition” is meant to encompass a composition suitable for administration to a subject. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or subjects in need thereof via a number of different routes of administration including oral, intravenous, buccal, rectal, parenteral, intraperitoneal, intradermal, intratracheal, intramuscular, subcutaneous, inhalational, and the like.

A “sustained reduction” of HDV viral load means a reduction of viral load (e.g., a decrease of at least 1.5 log₁₀ HDV RNA IU/mL serum, at least 2.0 log₁₀ HDV RNA copies/mL serum or at least 2.5 log₁₀ HDV RNA IU/mL serum, or a decrease in HDV RNA to undetectable levels) for a period time (e.g., 1 month, 3 months, 6 months, 1 year or longer). The sustained reduction may be a period of time during which the course of treatment is still ongoing or a period of time after the course of treatment is finished.

The term “therapeutically effective amount” as used herein refers to that amount of an embodiment of the agent (e.g., a compound, inhibitory agent, or drug) being administered that will treat to some extent a disease, disorder, or condition, e.g., relieve one or more of the symptoms of the disease, i.e., infection, being treated, and/or that amount that will prevent, to some extent, one or more of the symptoms of the disease, i.e., infection, that the subject being treated has or is at risk of developing.

The terms “treatment,” “treating,” and “treat” are defined as acting upon a disease, disorder, or condition with an agent to reduce or ameliorate the pharmacologic and/or physiologic effects of the disease, disorder, or condition and/or its symptoms. “Treatment,” as used herein, covers any treatment of a disease in a human subject, and includes: (a) reducing the risk of occurrence of the disease in a subject determined to be predisposed to the disease but not yet diagnosed as infected with the disease, (b) impeding the development of the disease, and/or (c) relieving the disease, i.e., causing regression of the disease and/or relieving one or more disease symptoms. “Treatment” is also meant to encompass delivery of an inhibiting agent to provide a pharmacologic effect, even in the absence of a disease or condition. For example, “treatment” encompasses delivery of an agent that provides for enhanced or desirable effects in the subject (e.g., reduction of viral load, reduction of disease symptoms, etc.).

The terms “undetectable” or “below the level of detection” or “BLD”, as used with reference to HDV RNA levels, means that no HDV RNA copies can be detected by the assay methodology employed. In some embodiments, the assay is quantitative RT-PCR.

The term “durable virologic response” or “DVR” as used herein refers to post-treatment response in a subject of HDV RNA below the limit of quantitation (BLQ) within one or more weeks after the end of treatment, or from between 2-12 weeks of ending treatment from between 12 and 24 weeks after ending treatment, or from 12-48 weeks after ending treatment.

### II. Methods of Treatment

In one aspect, the present disclosure provides methods of treating HDV infection by administering interferon lambda therapy to an HDV-infected subject. In some embodiments, a pegylated form of interferon lambda (e.g., pegylated interferon lambda-1a) is administered. In some embodiments, subjects receiving interferon lambda therapy (e.g., pegylated interferon lambda therapy) are also treated with an antiviral nucleoside or nucleotide analog (e.g., an anti-HBV nucleotide or nucleoside analog). In some embodiments, subjects receiving interferon lambda therapy (e.g., pegylated interferon lambda therapy) are also treated with lonafarnib therapy or lonafarnib and ritonavir therapy, e.g., for the duration of the interferon lambda therapy or during a portion of the time that interferon lambda therapy is administered. In some embodiments, subjects receiving interferon lambda therapy (e.g., pegylated interferon lambda therapy) are not administered an antiviral nucleoside or nucleotide analog therapy. In some embodiments, subjects receiving interferon lambda therapy (e.g., pegylated interferon lambda therapy) are not administered lonafarnib therapy or lonafarnib and ritonavir therapy.

### Interferon Lambda

Interferons are polypeptides that inhibit viral replication and cellular proliferation and modulate immune response. Based on the type of receptor through which they signal, human interferons have been classified into three major types (Types I, II, and III). All type I IFNs bind to a specific cell surface receptor complex known as the IFN-alpha receptor (IFNAR) that consists of IFNAR1 and IFNAR2 chains. The type I interferons present in humans are IFN-alpha, IFN-beta, IFN-epsilon, and IFN-omega. Type II IFNs bind to IFN-gamma receptor (IFNGR) that consists of IFNGR1 and IFNGR2 chains. The type II interferon in humans is IFN-gamma. The type III interferon group consists of three IFN-lambda molecules called IFN-lambda1, IFN-lambda2 and IFN-lambda3 (also called IL29, IL28A, and IL28B, respectively). These IFNs signal through a receptor complex consisting of IL10R2 (also called CRF2-4) and IFNLR1 (also called CRF2-12).

The term “interferon-lambda” or “IFN-λ” as used herein includes naturally occurring IFN-λ; synthetic IFN-λ; derivatized IFN-λ (e.g., PEGylated IFN-λ, glycosylated IFN-λ, and the like); and analogs of naturally occurring or synthetic IFN-λ. In some embodiments, an IFN-λ is a derivative of IFN-λ that is derivatized (e.g., chemically modified relative to the naturally occurring peptide) to alter certain properties such as serum half-life. As such, the term “IFN-λ” includes IFN-λ derivatized with polyethylene glycol (“PEGylated IFN-λ”), and the like. PEGylated IFN-λ (e.g., PEGylated IFN-λ-1a), and methods for making same, is discussed in, e.g., U.S. Pat. Nos. 6,927,040, 7,038,032, 7,135,170, 7,157,559, and 8,980,245; and PCT publication Nos. WO 2005/097165, WO 2007/012033, WO 2007/013944 and WO 2007/041713; all of which are herein incorporated by reference in their entirety. In some embodiments, the IFN-λ is an IFN-λ as disclosed in PCT/US2017/018466, which is incorporated by reference herein in its entirety. In some embodiments, the pegylated IFN-λ-1a has the structure described in U.S. Pat. No. 7,157,559, which is incorporated by reference herein in its entirety.

In some embodiments, an interferon for use in a therapeutic method as described herein is a pegylated IFN-λ1 (e.g., pegylated IFN-λ-1a), pegylated IFN-λ-2, or pegylated IFN-λ-3. In some embodiments, the interferon is pegylated IFN-λ1 (e.g., pegylated IFN-λ-1a).

In some embodiments, pegylated IFN-λ1 has the amino acid sequence shown below (lines show intrachain disulfide bonds)[SEQ ID NO:1]:

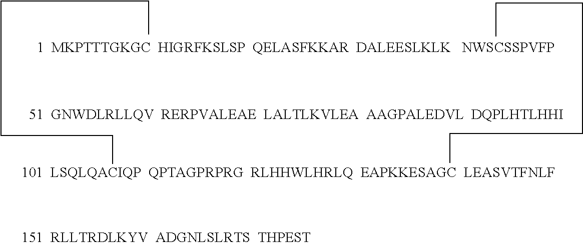

### Subject Population

In some embodiments, a subject to be treated with interferon lambda therapy as described herein is a subject having an HDV infection, an acute HDV infection, or a chronic HDV infection. In some embodiments, the subject to be treated has a chronic HDV infection of at least 6 months' duration documented by a positive HDV antibody (Ab) test, and/or detectable HDV RNA by qRT-PCR. In some embodiments, a subject to be treated with a therapeutic method described herein is a subject having an acute HDV infection, one that is newly diagnosed or otherwise believed not to have existed in the subject for more than six months. Diagnosis and pathogenesis of HDV is described, for example, in Wedemeyer et al., Nat. Rev. Gastroenterol. Hepatol, 2010, 7:31-40. HDV is known to exist in a variety of subtypes; the methods described herein are suitable for treating all HDV subjects, regardless of HDV subtype. In some embodiments, the subject is an adult (18 years or older) and in other embodiments, the subject is pediatric.

In some embodiments, a subject to be treated has a baseline viral load of at least 10² HDV RNA copies per mL serum or plasma or at least 10² IU/mL serum or plasma, e.g., at least 10³ HDV RNA copies per mL or at least 10³ IU/mL serum or plasma, at least 10⁴ HDV RNA copies per mL or at least 10⁴ IU/mL serum or plasma, at least 10⁵ HDV RNA copies per mL or at least 10⁵ IU/mL serum or plasma, at least 10⁶ HDV RNA copies per mL or at least 10⁶ IU/mL serum or plasma, at least 10² HDV RNA copies per mL or at least 10² IU/mL serum or plasma, or at least 10⁸ HDV RNA copies per mL or at least 10⁸ IU/mL serum or plasma. In some embodiments, HDV viral load is measured using serum samples from the subject. In some embodiments, HDV viral load is measured using plasma samples from the subject. In some embodiments, viral load is measured by quantitative RT-PCR. qRT-PCR assays for quantification of HDV RNA in serum or plasma are known in the art, e.g., as described above. In some embodiments, a subject to be treated has a baseline viral load that is up to about 10⁴ HDV RNA copies per mL serum or plasma or up to about 10⁴ IU/mL serum or plasma. In some embodiments, a subject to be treated has a baseline viral load that is up to about 10⁵ HDV RNA copies per mL serum or plasma or up to about 10⁵ IU/mL serum or plasma. In some embodiments, a subject to be treated has a baseline viral load that is up to about 10⁶ HDV RNA copies per mL serum or plasma or up to about 10⁶ IU/mL serum or plasma.

In some embodiments, HDV viral load is measured using serum samples from the subject. In some embodiments, HDV viral load is measured using plasma samples from the subject. In some embodiments, viral load is measured by quantitative RT-PCR. qRT-PCR assays for quantification of HDV RNA in serum or plasma are known in the art, e.g., as described above.

In some embodiments, a subject to be treated exhibits one or more symptoms of liver dysfunction. In some embodiments, the subject exhibits one or more liver function parameters that are outside the normal parameters for a healthy control (e.g., a subject that is not infected with HDV or HBV). In some embodiments, the liver function parameter is selected from the group consisting of serum albumin, bilirubin, alanine aminotransferase (ALT), aspartate aminotransferase (AST), and prothrombin activity. In some embodiments, the subject has a serum ALT level that is at least two-fold higher than the upper limit of normal (ULN) (e.g., at least 2-fold, at least 3-fold, at least 4-fold, at least 5-fold, at least 6-fold, at least 7-fold, at least 8-fold, at least 10-fold or higher than the ULN). Liver function parameters are described in the art. See, e.g., Limdi et al., Postgrad Med J, 2003, 79:307-312. Methods of measuring these liver function parameters are known in the art and are also commercially available.

In some embodiments, the subject has compensated liver disease (e.g., as classified according to the Child-Turcotte-Pugh Classification System) with or without liver cirrhosis. It will be recognized by a person of ordinary skill in the art that the Child-Turcotte-Pugh Classification System is used to classify the severity of liver disease and is determined by assessing serum albumin levels, bilirubin levels, international normalized ratio of prothrombin time levels, ascites formation, and encephalopathy. In some embodiments, the subject has a Child-Turcotte-Pugh score of 5-6 (class A). In some embodiments, the subject has a Child-Turcotte-Pugh score of 1-6. In some embodiments, the subject has a Child-Turcotte-Pugh score of 1-2, or 1-3, or 2-4, or 3-4, or 2-5, or 3-5 or 2-6. In some embodiments, the subject has compensated liver disease with liver cirrhosis. In some embodiments, the subject has compensated liver disease without liver cirrhosis.

In some embodiments, the subject is diagnosed with chronic hepatitis as determined by, for example, one or more of: liver biopsy, liver function test, ultrasound, hepatic venous pressure gradient (HVPG) measurement, ALT level, other blood tests, or albumin level. In some embodiments, the biopsy is within the 6 months before treatment. In some embodiments, the biopsy is within the 18 months before initiation of treatment according to the methods provided herein. In some embodiments, the biopsy is within the 1 day to 24 months before treatment. In some embodiments, the subject has evidence of chronic hepatitis based on a liver biopsy within 6 months before screening. In some embodiments, the subject has a serum alanine aminotransferase (ALT) level that is above the upper limit of normal (ULN) within 24 weeks prior to treatment and/or at the initiation of treatment, within 24 months prior to treatment, 24 months-1 month prior to treatment, or within 12 months to 1 day prior to treatment. In various embodiments, the subject meets one or more independently selected eligibility criteria in Example 1.

### Interferon Lambda Dosing Regimens

In some embodiments, interferon lambda therapy comprises administering to the subject interferon lambda (e.g., pegylated interferon lambda-1a) at a dose of 180 micrograms (mcg) per week, 120 mcg per week, 110 mcg per week, 100 mcg per week, 90 mcg per week, 80 mcg per week, 120-70 mcg per week, 200-120 mcg per week, 170-130 mcg per week. In some embodiments, interferon lambda is administered at a dose of 180 mcg QW. In some embodiments, interferon lambda is administered at a dose of 90 mcg two time per week. In some embodiments, interferon lambda is administered at a dose of 90 mcg every 3-4 days. In some embodiments, interferon lambda is administered at a dose of 80 mcg two time per week. In some embodiments, interferon lambda is administered at a dose of 80 mcg every 3-4 days. In some embodiments, interferon lambda is administered at a dose of 100-70 mcg two time per week. In some embodiments, interferon lambda is administered at a dose of 100-70 mcg every 3-4 days. In some embodiments, interferon lambda is administered at a dose of 120 mcg QW. In some embodiments, interferon lambda is administered at a dose of 80 mcg QW.

In some embodiments, a subject being treated for HDV infection receives an adjustment in the dosing regimen of the interferon lambda therapy during the course of treatment. In some embodiments, the subject receives a dose reduction of interferon lambda, in that one or more later doses is a lower dose than one or more earlier doses. In some embodiments, a dose is reduced if the subject exhibits unacceptable side effects. In some embodiments, a subject may receive multiple dose reductions during the course of treatment with interferon lambda. In some embodiments, the dosage administered to the subject is not reduced before 8 weeks of treatment at the first dosage (e.g., at a first dosage of 180 mcg QW), or before 1 week, or 2 weeks, or 3 weeks, or 4 weeks, or 5 weeks, or 6 weeks, or 7 weeks of treatment at the first dosage. In some embodiments, the dosage administered to the subject is not reduced before 9-12 weeks of treatment at the first dosage (e.g., at a first dosage of 180 mcg QW).

In some embodiments, the interferon lambda therapy comprises administering to the subject interferon lambda at a dose of 180 micrograms per week for a first treatment period followed by administering to the subject interferon lambda at a dose of 120 micrograms per week for a second treatment period. In some embodiments, the length of time for the first treatment period is the same as the length of time for the second treatment period. In some embodiments, the first treatment period and the second treatment period are different lengths of time. In some embodiments, the first treatment period (i.e., interferon lambda at a dose of 180 mcg per week) is longer than the second treatment period (i.e., interferon lambda at a dose of 120 mcg per week). In some embodiments, the second treatment period (i.e., interferon lambda at a dose of 120 mcg per week) is longer than the first treatment period (i.e., interferon lambda at a dose of 180 mcg per week). In some embodiments, the interferon lambda therapy further comprises administering to the subject interferon lambda at a dose of 110-80 micrograms per week for a third treatment period. In some embodiments, the length of time for the third treatment period is the same as the length of time for the first and/or second treatment period. In some embodiments, the third treatment period and the first and/or second treatment period are different lengths of time. In some embodiments, the third treatment period (i.e., interferon lambda at a dose of 110-80 mcg per week) is longer than the first and/or second treatment period. In some embodiments, the third treatment period (i.e., interferon lambda at a dose of 80 mcg per week) is shorter than the first and/or second treatment period.

In some embodiments, the interferon lambda therapy comprises administering interferon lambda at a dose of 120 micrograms per week for a first treatment period followed by administering interferon lambda at a dose of 110-80 micrograms per week for a second treatment period. In some embodiments, the length of time for the first treatment period is the same as the length of time for the second treatment period. In some embodiments, the first treatment period and the second treatment period are different lengths of time. In some embodiments, the first treatment period (i.e., interferon lambda at a dose of 120 mcg per week) is longer than the second treatment period (i.e., interferon lambda at a dose of 80 mcg per week). In some embodiments, the second treatment period (i.e., interferon lambda at a dose of 80 mcg per week) is longer than the first treatment period (i.e., interferon lambda at a dose of 120 mcg per week).

In some embodiments, the interferon lambda therapy comprises administering interferon lambda at a first dose of 180 micrograms QW for a first treatment period, at a second dose of 170-120 micrograms QW for a second treatment period, and at a third dose of 110-80 micrograms QW for a third treatment period. In some embodiments, the first treatment period has a duration of at least 8 weeks, or from 1-8 weeks, or from 1-12 weeks. In some embodiments, the first treatment period has a duration of 8-12 weeks.

In some embodiments, the interferon lambda therapy comprises administering interferon lambda at a first dose of 160-180 micrograms per week for a first treatment period, at a second dose of 170-120 micrograms per week for a second treatment period, and at a third dose of 110-60 micrograms per week for a third treatment period. In some embodiments, the first treatment period has a duration of at least 8 weeks, or from 1-8 weeks, or from 1-12 weeks. In some embodiments, the first treatment period has a duration of 8-12 weeks. Doses may be given in multiple dose per week with the number of micrograms equaling the weekly dose.

In some embodiments, a treatment period (e.g., a first treatment period, second treatment period, and/or third treatment period) is at least 1 week in duration, e.g., at least 2, 3, 4 weeks or longer. In some embodiments, a treatment period (e.g., a first treatment period, second treatment period, and/or third treatment period) is at least 2 weeks in duration, e.g., at least 4, 6, 8, 10, 12, 16, 20, 24, 28, 32, 36, 40, 44, 48 weeks, or longer. In some embodiments, a treatment period is at least 8 weeks in duration. In some embodiments, a treatment period is up to about 4 weeks in duration, or up to about 6, 8, 10, 12, 16, 20, 24, 28, 32, 36, 40, 44, or 48 weeks in duration. In some embodiments, a treatment period is up to about 8 weeks in duration. In some embodiments, a treatment period is up to about 12 weeks in duration.

For a subject receiving a dose reduction, in some embodiments, a treatment period at a first dose is paused or stopped prior to starting a subsequent treatment period at a second lower dose. For example, in some embodiments, a first treatment period (e.g., at a dose of 180 mcg per week) is paused or stopped for a period of at least 1 week, 2 weeks, 3 weeks, 4 weeks or longer prior to starting a second treatment period (e.g., at a dose of 120 mcg per week).

In some embodiments, a subject is administered a first dose of 180 micrograms QW for at least 8 weeks before there is a dose reduction. In some embodiments, a subject is administered a first dose of 180 micrograms QW for at least 8-12 weeks before there is a dose reduction.

In some embodiments, if the subject has an absolute neutrophil count (ANC) of between ≥to 500/mm³ and <750/mm³, or between ≥to 400/mm³ and <650/mm³, or between to ≥400/mm³ and <850/mm³, the subject will begin the second treatment period.

In some embodiments, if the subject has an ANC of <500/mm³, dosing of the subject will stop until the subject has an ANC of >1000/mm³ and then dosing will be resumed for a second treatment period. In another embodiment, if the subject has an ANC of <400/mm³, dosing of the subject will stop until the subject has an ANC of >750/mm³ and then dosing will be resumed for a second treatment period.

In some embodiments, if the subject has a platelet level of <50,000 then subject will begin the second treatment period or if a subject has a platelet level of <25,000 then subject will discontinue treatment.

In some embodiments, if the subject has a total bilirubin (TBILI)>2.5×upper limit of the normal range (ULN) and direct bilirubin (DB)>3×ULN, dosing of the subject will stop until the subject has a TBILI≤1.5×ULN and then dosing will resume for a second treatment period.

In some embodiments, if the subject has a TBILI>3×ULN and DB>3×ULN, dosing of the subject will be interrupted until the TBILI≤1.5×ULN and then dosing will resume for a second treatment period.

In some embodiments, if the subject has an ALT (or AST)≥20×ULN and TBILI and/or international normalized ratio (INR)<Grade 2, dosing of the subject will be interrupted until the ALT/AST<10×ULN and then dosing will resume for a second treatment period. In some embodiments, if the subject has an absolute neutrophil count (ANC) of alanine aminotransferase (ALT) (or aspartate aminotransferase (AST))≥20×ULN and TBILI and/or INR<Grade 2 for a second time, dosing of the subject will be interrupted and then dosing will resume for a second treatment period.

In some embodiments. if the subject has an ALT (or AST)≥15-20×ULN and TBILI and/or INR<Grade 2, dosing of the subject will be interrupted dosing until the ALT/AST<10×ULN and then dosing will resume for a second treatment period; or if the subject has an ANC of ALT (or AST)≥15-20×ULN and TBILI and/or INR<Grade 2 for a second time, dosing of the subject will interrupt dosing until the ALT/AST<10×ULN and then dosing will resume for a second treatment period.

In some embodiments, the dose resumption after an interruption or stopping is resumed one week, two weeks, three weeks or four weeks after the interruption on stopping.

In some embodiments, if the subject has an ALT (or AST)≥15×ULN and TBILI and/or INR<Grade 2, dosing of the subject will be interrupted until the ALT/AST<10×ULN and then dosing will resume for a second treatment period. In some embodiments, if the subject has an ANC of ALT (or AST)≥15×ULN and TBILI and/or INR<Grade 2 for a second time, dosing of the subject will be interrupted and then dosing will resume for a second treatment period.

In some embodiments, if the subject has an ALT (or AST)≥5×ULN and TBILI and/or INR≥Grade 2, treatment of the subject will terminate.

In some embodiments, if the subject has an ALT (or AST)≥10×ULN and TBILI and/or INR≥Grade 3, treatment of the subject will terminate.

In some embodiments, if the subject experiences an adverse event≥Grade 3, dosing of the subject will stop until the event resolves or is ≤a Grade 1 and the dosing will resume for a second treatment period.

In some embodiments, if the subject experiences a second adverse event of ≥Grade 3, dosing of the subject will be interrupted and then resume dosing for a third treatment period.

In some embodiments, if a subject has a creatinine clearance level of <50 mL/min, treatment of the subject is discontinued.

In some embodiments, subjects who meet the criteria for treatment interruption, reduction and/or discontinuation, based on hepatobiliary abnormalities may have a clinical work-up that includes one or more of the following: autoimmune markers (antinuclear antibody [ANA], anti-smooth muscle antibody [e.g., anti-SMA], anti-LC1, anti-SLA liver kidney microsome type 1 and type III antibody [e.g., anti-LKM1,3]); C3, C4 and CH50; acute viral hepatitis; Serologies for acute hepatitis A and E (IgM); PCR for HCV, hepatitis E (stool and blood); cytomegalovirus (CMV), Epstein-Barr virus (EBV), or Herpes simplex viruses 1 and 2 (HSV), for example, by PCR; cholestasis work up with a Doppler US of the liver; review of pre-existing hepatic disease (excluding HBV); review of concomitant medication(s), or herbal medications and substances known to be hepatotoxic, tests for alcohol and acetaminophen and drugs of abuse, if indicated; ultrasound of the liver should be performed, including doppler, for subjects with a bilirubin level greater than 1.5 times baseline; if clinically feasible, a liver biopsy should be performed; when a percutaneous biopsy is contraindicated, a transjugular biopsy may be discussed; liver and chemistry labs should be performed weekly (minimally include ALT, AST, bilirubin, INR, alkaline phosphatases and gamma-GT) until the bilirubin returns to baseline value; HBV DNA and HDV RNA should be monitored weekly until Bili<1.5×ULN; 5 ml of serum plus 5 ml of plasma should be collected for possible later biomarker analysis.

In certain embodiments, subjects with a 4× increase in baseline GGT, ALT/AST or alkaline phosphatases or >Bili 1.5 mg/dL, direct Bilirubin>0.6 (if Gilbert Syndrome is present) during any treatment period, may be prescribed ursodeoxycholic acid for “liver protection”.

In certain embodiments, the subject is also administered Tenofovir DF or entecavir for treatment of hepatitis B.

In some embodiments, subjects with Stage 0-III disease, wherein the subject has a ≤11.4 kPa at baseline, and requires a dose interruption, then reductions then discontinuations are in sequence as follows: in subjects with Alb>3.5 g/dL and INR<1.5 and total Bilirubin<3 mg/dL, then dose interruption 1 at ALT>20×ULN (>1000 IU/mL) and restart at next lower lambda dose when ALT<10×ULN (<500 IU/mL), and next dose interruption 2 at ALT 20×ULN (>1000 IU/mL), then restart at next lower lambda dose when ALT<10×ULN (<500 IU/mL) or stop if at 80 mg dose bilirubin is 3.0 mg/dL or greater.

As used herein, the following abbreviations are used: AE, adverse event; ALT, alanine aminotransferase; ANC, absolute neutrophil count; AST, aspartate aminotransferase; CTCAE, Common Terminology Criteria for Adverse Events; DB, direct bilirubin; DILI, drug-induced liver injury; PT, prothrombin time; SAE, serious adverse event; TBILI, total bilirubin; ULN, upper limit of the normal range.

### Duration of Treatment and Treatment Endpoints

Subjects may receive interferon lambda therapy for a predetermined time, an indefinite time, or until an endpoint is reached. Treatment may be continued on a continuous daily basis for at least two to three months. In some embodiments, therapy is for at least 30 days, at least 60 days, at least 90 days, at least 120 days, at least 150 days, or at least 180 days. In some embodiments, treatment is continued for at least 6 months, at least 7 months, at least 8 months, at least 9 months, at least 10 months, at least 11 months, at least one year, at least 15 months, at least 18 months, or at least 2 years. In some embodiments, therapy is for at least 6 weeks, 12 weeks, 18 weeks, 24 weeks, 30 weeks, 36 weeks, 42 weeks, 48 weeks, 60 weeks, 72 weeks, 84 weeks, or 96 weeks. In other embodiments, treatment is continued for the rest of the subject's life or until administration is no longer effective in maintaining the virus at a sufficiently low level to provide meaningful therapeutic benefit.

In accordance with the methods herein, some HDV subjects will respond to therapy as described herein by clearing virus to undetectable levels. In some embodiments, for subjects in which HDV RNA levels are below the level of detection, treatment is suspended unless and until the HDV levels return to detectable levels. Other subjects will experience a reduction in viral load and improvement of symptoms but will not clear the virus to undetectable levels but will remain on therapy for a defined period of time (e.g., for about 1 year, about 2 years, about 3 years, or longer) or so long as it provides therapeutic benefit.

In some embodiments, treatment with interferon lambda therapy results in a reduction of HDV viral load in the subject of at least 1.5 log₁₀ HDV RNA copies/mL serum when measured after 48 weeks of treatment. In some embodiments, treatment with interferon lambda therapy results in a reduction of HDV viral load in the subject of at least 2.0 log₁₀ HDV RNA copies/mL serum when measured after 48 weeks of treatment. In some embodiments, treatment with interferon lambda therapy results in a reduction of HDV viral load in the subject of at least 2.5 log₁₀ HDV RNA copies/mL serum when measured after 48 weeks of treatment.

In some embodiments, treatment with interferon lambda therapy results in a sustained reduction of HDV viral load (e.g., a decrease of at least 1.5 log₁₀ HDV RNA IU/mL serum, at least 2.0 log₁₀ HDV RNA copies/mL serum or at least 2.5 log₁₀ HDV RNA IU/mL serum, or a decrease in HDV RNA to undetectable levels) that is sustained for a period of time (e.g., 1 month, 3 months, 6 months, 1 year or longer) while the course of treatment is still ongoing. In some embodiments, treatment with interferon lambda therapy results in a sustained reduction of HDV viral load that is sustained for a period of time (e.g., 1 month, 3 months, 6 months, 1 year or longer) after the course of treatment is finished. In some embodiments, the course of treatment results in HDV RNA levels (e.g., serum HDV RNA levels or plasma HDV RNA levels) below 1,000 copies/mL. In some embodiments, the HDV RNA levels remain below 1,000 copies/mL for at least one month, at least three months, at least one year, or longer. In some embodiments, the course of treatment results in HDV RNA levels (e.g., serum HDV RNA levels or plasma HDV RNA levels) below 100 copies/mL. In some embodiments, the HDV RNA levels remain below 100 copies/mL for at least one month, at least three months, at least one year, or longer. The phrase “remains below” refers to remaining below an initial measured value (e.g., 100 copies/mL or 100 IU/mL) for a period of time, for example, at 1 month (or another specified time) a viral load measurement taken at least 1 month (or at the other specified time) after determination of the initial measured value is no higher than the initial value. In some embodiments, the subject does not receive interferon lambda therapy during the specified time. In some embodiments, the subject does not receive any anti-HDV treatment during the specified time.

In some embodiments, therapy as disclosed herein is continued for a period of time until HDV RNA levels are below 3 log₁₀ HDV RNA copies/mL (below 1,000 copies/mL), or sometimes until HDV RNA levels are below 2 log₁₀ HDV RNA copies/mL (below 100 copies/mL) or below the level of detection. In some embodiments, therapy is continued for a period of time (such as 1 to 3 months or longer) after viral load has dropped to acceptably low levels (e.g., undetectable levels). In some embodiments, therapy is continued until the HDV viral load is reduced to undetectable levels.

In some embodiments, a subject treated according to the methods described herein exhibits a reduction in HDV viral load to undetectable levels during the course of treatment, and the subject maintains the reduction in HDV viral load to undetectable levels for at least 12 weeks after the end of treatment. In some embodiments, a subject treated according to the methods described herein exhibits a reduction in HDV viral load to undetectable levels during the course of treatment, and the subject maintains the reduction in HDV viral load to undetectable levels for at least 24 weeks after the end of treatment.

In some embodiments, the subject's HDV titer rises from baseline prior to dropping below baseline during the course of treatment. In some embodiments, the subject's HDV level rises to more than 150% of baseline, or more than 200% of baseline. In some embodiments, the rise in the titer is between 25-50% of baseline, or from 25-100% of baseline, or from 50-200% of baseline. In some embodiments, the rise in the titer occurs within 2 weeks after initiation of therapy. In some embodiments, the subject's elevated HDV titer drops to below baseline within 2 weeks, or within 3 weeks, of initiation of therapy.

In some embodiments, a subject treated according to the methods described herein exhibits an improvement in one or more liver function parameters. In some embodiments, the improved liver function is an improvement in one or more serum markers (e.g., one, two, three, four, five, six or more markers), such as serum albumin, bilirubin, alanine aminotransferase (ALT), aspartate aminotransferase (AST), prothrombin, alfa2-macroglobulin, apolipoproteinAl, haptoglobin, gamma-glutamyl transpeptidase (GGT). In some embodiments, a subject treated according to the methods described herein exhibits an improvement in liver fibrosis (e.g., as assessed by biopsy with histological analysis, transient ultrasound elastography (e.g., FibroScan), or magnetic resonance elastography). In some embodiments, treatment results in an improvement of at least 5%, at least 10%, at least 20%, at least 30%, at least 40%, at least 50%, at least 70%, at least 75%, at least 80%, at least 100% or between 5-50%, or between 10-80%, or between 50-100% improvement in one or more liver function parameters (e.g., an improvement in serum marker(s) or an improvement in liver fibrosis) in the subject as compared to prior to the onset of treatment. In some embodiments, treatment results in an improvement in one or more liver function parameters (e.g., an improvement in serum marker(s) or an improvement in liver fibrosis) to the level of a healthy control subject that is not infected with HDV or HBV. In some embodiments, the subject exhibits an improvement in serum ALT levels to a level that is within the upper limit of normal.

In some embodiments, a subject treated according to the methods described herein exhibits a reduction in HBV viral load compared to the baseline level at the initiation of treatment and/or compared to a similarly infected subject not receiving treatment effective to reduce the subject's HDV viral load. In some embodiments, treatment results in a reduction of at least 1 log₁₀ in HBV viral load.

In some embodiments, a subject treated according to the methods described herein exhibits an improvement in one or more parameters described in Example 1. In some embodiments, subjects treated according to the methods of the invention exhibit a reduction in HDV and/or HBV viral load. Prior to treatment, the subject's HDV and/or HBV viral load is measured to determine the baseline viral load. After a period of treatment (e.g., after 12 weeks of treatment), the subject's viral load is reduced compared to baseline. In some embodiments, after a period of treatment (e.g., after 12 weeks of treatment), the subject's viral load is substantially reduced compared to baseline, such as to very low levels or to an undetectable level. In some embodiments, treatment results in an at least 2 log₁₀ reduction of HBV viral load. In some embodiments, subjects treated according to the methods described herein exhibit a reduction in HBsAg levels or an improvement in clearance of HBsAg antigen. Prior to treatment the subject's HBsAg level is measured to determine a baseline. After a period of treatment (e.g., after 12 weeks of treatment), the subject's HBsAg level is reduced compared to baseline. In some embodiments, subjects treated according to the methods described herein exhibits the presence of anti-HBs antibody.

In one embodiment, in a population of subjects treated with 180 μg of interferon lambda, response rates differed between subjects with high (>4 log₁₀) versus low (≤log₁₀) baseline viral load. In one embodiment, at week 48, 38-43% and 33-40% of subjects with high versus low baseline viral loads respectively, reached HDV RNA levels BLQ. In another embodiment, at week 72, the difference between these two groups became more prominent, with 50-60% of subjects in the low baseline viral load reaching BLQ versus 25-29% in the high baseline viral load meeting this endpoint.

In one embodiment, the subject has a chance of between about 11% to about 14% in treatment resulting in the ALT levels normalizing. In another embodiment, the subject has about a 12.1% to about 42.4% chance of the treatment resulting in the 2 log₁₀ or greater decline in HDV RNA. In another embodiment, the subject has a 15/1% to about 39.4% chance of the treatment resulting in the HDV RNA being BLQ. In another embodiment, a viral load decline between about −1.18 log₁₀ HDV RNA and about −2.35 log₁₀ HDV RNA is observed at 48 weeks of treatment. In one embodiment, the subject has an increased chance of achieving ALT normalization and a >2 log₁₀ decline after a last administration than during administration and, in some instances, the last administration is between week 4 and week 48 of administration. In another embodiment, the subject has a chance of about 36-45% of the treatment resulting in ALT normalization at 24-weeks post-dosing when administered 180 mcg/week. In one embodiment, the subject has a chance of between about 26-36% of subject having a reduction to a second dose during treatment; between about 5-9% chance of having a dose interruptions, or between about 21-26% chance of discontinuing treatment.

In one embodiment, the percent of subjects being administered the 180 mcg/week dose have one or more of the following: dose reductions (about 30-36%), interruptions (about 7-9%), and treatment discontinuations (about 21-24%). In another embodiment, the percent of subjects being administered the 120 mcg/week dose have one or more of the following: dose reductions (about 26-30%), interruptions (about 5-9%), and treatment discontinuations (about 24-26%). In another embodiment, wherein 38-43% subjects receiving a starting dose of 180 micrograms per week and after a last administration who had a high (>4 log₁₀) baseline viral load achieved HDV RNA levels BLQ at week 48. In another embodiment, 25-29% subjects receiving a starting dose of 180 micrograms per week and after a last administration who had a high (>4 logs) baseline viral load achieved HDV RNA levels BLQ at 24 weeks post treatment. In another embodiment, 33-40% subjects receiving a starting dose of 180 micrograms per week and after a last administration who had a low (≤4 log₁₀) baseline viral load achieved HDV RNA levels BLQ at week 48. In another embodiment, 50-60% subjects receiving a starting dose of 180 micrograms per week and after a last administration who had a low (≤4 log₁₀) baseline viral load achieved HDV RNA levels BLQ at 24 weeks posttreatment. In another embodiment, 25-29% subjects receiving a starting dose of 180 micrograms per week and after a last administration who had a high (>4 log₁₀) baseline viral load achieved undetectable HDV RNA levels at week 48 and 24 weeks posttreatment. In another embodiment, 33-40% subjects receiving a starting dose of 180 micrograms per week and after a last administration who had a low (≤log₁₀) baseline viral load achieved undetectable HDV RNA levels at week 48 and 24 weeks post treatment.

In another embodiment, after a last dose one or more of: 16-21% subjects receiving a starting dose of 120 micrograms per week achieved HDV RNA levels BLQ; 21-29% subjects receiving a starting dose of 120 micrograms per week achieved >2 log₁₀ decline; 11-14% subjects receiving a starting dose of 120 micrograms per week achieved ALT normalization; 5-7% subjects receiving a starting dose of 120 micrograms per week achieved ALT Normalization+>2 log₁₀ decline. In another embodiment, 24 weeks after a last dose one or more of: 16-21% subjects receiving a starting dose of 180 micrograms per week achieved HDV RNA levels BLQ; 11-14% subjects receiving a starting dose of 180 micrograms per week achieved >2 log₁₀ decline; 26-36% subjects receiving a starting dose of 180 micrograms per week achieved ALT normalization; 11-14% subjects receiving a starting dose of 180 micrograms per week achieved ALT Normalization+>2 log₁₀ decline. In another embodiment, after a last dose one or more of: 36-45% subjects receiving a starting dose of 180 micrograms per week achieved HDV RNA levels BLQ; 50-64% subjects receiving a starting dose of 180 micrograms per week achieved >2 log 10 decline; 14-18% subjects receiving a starting dose of 180 micrograms per week achieved ALT normalization; 14-18% % subjects receiving a starting dose of 180 micrograms per week achieved ALT Normalization+>2 log₁₀ decline. In another embodiment, 24 weeks after a last dose one or more of: 36-45% subjects receiving a starting dose of 180 micrograms per week achieved HDV RNA levels BLQ; 36-45%% subjects receiving a starting dose of 180 micrograms per week achieved >2 log₁₀ decline; 36-45%% subjects receiving a starting dose of 180 micrograms per week achieved ALT normalization; 29-36% subjects receiving a starting dose of 180 micrograms per week achieved ALT Normalization+>2 log₁₀ decline.

### Antiviral Co-Therapy

In some embodiments, a subject who is administered interferon lambda therapy according to the present disclosure may also be treated with one or more other antiviral agents such as nucleoside and nucleotide analogs, compounds used to treat HBV infections, and other agents.

In some embodiments, a subject who is administered interferon lambda therapy is treated with an antiviral agent that is used for the treatment of HBV. Anti-HBV medications that are currently approved, with the exception of interferons, inhibit reverse transcriptase and are nucleoside or nucleotide analogs. These medications, while effective against HBV, are not effective against HDV as they do not clear HBsAg, which HDV needs to replicate. Currently approved anti-HBV nucleoside/nucleotide analogs include lamivudine (Epivir-HBV®, Zeffix®, or Heptodin®), adefovir dipivoxil (Hepsera®), entecavir (Baraclude®), telbivudine (Tyzeka® or Sebivo®), clevudine (Korea/Asia), tenofovir (Viread® or Vemlidy). In some embodiments, a subject who is administered interferon lambda therapy is also administered a nucleoside or nucleotide analogs, such as but not limited to lamuvidine, adefovir, telbivudine, entecavir, tenofovir, or clevudine. In some embodiments, the subject is receiving nucleoside or nucleotide analog therapy prior to the onset of interferon lambda therapy. In some embodiments, nucleoside or nucleotide analog therapy is initiated at the start of interferon lambda therapy or during the course of interferon lambda therapy.

In some embodiments, a subject who is administered interferon lambda therapy is treated with lonafarnib. Lonafarnib therapy for the treatment of HDV is disclosed in US 2017/0042862, incorporated by reference herein. In some embodiments, a subject who is administered interferon lambda therapy also receives lonafarnib therapy at a total daily dose of 50-200 mg per day, e.g., 50 mg per day, 75 mg per day, 100 mg per day, 150 mg per day, or 200 mg per day. Lonafarnib therapy may be administered once daily (QD) or twice daily (BID). In some embodiments, a subject who is administered interferon lambda therapy also receives lonafarnib therapy at a dose of 25 mg BID, 50 mg BID, 75 mg BID, 100 mg BID, 50 mg QD, 75 mg QD, or 100 mg QD. In some embodiments, lonafarnib therapy is initiated at the start of interferon lambda therapy or during the course of interferon lambda therapy.

In some embodiments, a subject who is administered interferon lambda therapy is treated with lonafarnib and CYP3A inhibitor co-therapy, such as ritonavir or cobicistat. In some embodiments, the CYP3A inhibitor is ritonavir. Lonafarnib and ritonavir co-therapy is disclosed in WO 2015/168648 and in WO 2017/079009, incorporated by reference herein. In some embodiments, a subject who is administered interferon lambda therapy also receives lonafarnib-ritonavir co-therapy at a total daily dose of 50-200 mg of lonafarnib per day (e.g., 50 mg per day, 75 mg per day, 100 mg per day, 150 mg per day, or 200 mg per day of lonafarnib) and 100-200 mg of ritonavir per day (e.g., 100 mg per day, 150 mg per day, or 200 mg per day of ritonavir). Lonafarnib-ritonavir co-therapy may be administered once daily (QD) or twice daily (BID). In some embodiments, a subject who is administered interferon lambda therapy also is administered lonafarnib at a dose of 25 mg BID, 50 mg BID, 75 mg BID, 100 mg BID, 50 mg QD, 75 mg QD, or 100 mg QD, and ritonavir at a dose of 50 mg BID or 100 mg BID. In some embodiments, lonafarnib-ritonavir co-therapy is initiated at the start of interferon lambda therapy or during the course of interferon lambda therapy.

Other therapeutic compounds that may be administered with beneficial effect to an HDV-infected subject that is receiving interferon lambda therapy include a thiazolide; a protease inhibitor; a polymerase inhibitor; a helicase inhibitor; a Class C CpG toll-like receptor 7 and/or 9 antagonist; an amphipathic helix disruptor or NS4B inhibitor; a statin or other HMG CoA reductase inhibitor; an immunomodulator; an anti-inflammatory; a second prenylation inhibitor; a cyclophilin inhibitor; and an alpha-glucosidase inhibitor.

In various embodiments, interferon lambda is administered with standard nucleoside HBV medications as well as the promising new anti-HDV therapy described in US 2017/0042862, e.g., lonafarnib therapy, optionally administered in combination with a boosting agent such as ritonavir, for optimum therapeutic efficacy. When administered in combination with other anti-HBV or HDV drugs, the physician may, in accordance with the invention, initiate dosing of interferon lambda at any daily dose in the general range of 80-180 mcg, with starting doses of, for example, 180, 120, or 80 mcg/day. Some subjects of Pakistani origin have exhibited undesired side effects (high bilirubin levels) from interferon lambda therapy as described herein, possibly due to genetic variations in genes affecting bilirubin transport, and the physician may wish to pursue other treatment regimens first and in any event monitor such subjects closely for the appearance of such side effects to ensure dosing is discontinued or reduced to eliminate them.

### Formulation and Administration

Interferon lambda may be formulated for administration by any therapeutically appropriate route. In some embodiments, interferon lambda is formulated for administration by intravenous or subcutaneous administration. Other routes suitable for drug delivery, including systemic and localized routes of administration, may be used.

In some embodiments, interferon lambda is administered by subcutaneous administration (e.g., subcutaneous injection). Sites of injection, include, but are not limited to, injection in the thigh, abdomen, upper arm region, or upper buttock region.

In embodiments, interferon lambda (e.g., pegylated interferon lambda) is provided as a pharmaceutical formulation comprising the interferon lambda and one or more excipients such as preservatives, surfactants (e.g., a polysorbate or a poloxamer), or colorants (e.g., pharmaceutically acceptable dyes, inorganic pigments, and natural colorants). A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc., each of which is incorporated by reference herein.

In some embodiments, interferon lambda can be formulated into a preparation for injection by dissolving, suspending or emulsifying the interferon lambda in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Unit dosage forms for injection or intravenous administration may comprise in a composition as a solution in sterile water, normal saline or another pharmaceutically acceptable carrier. Appropriate amounts of the active pharmaceutical ingredient for unit dose forms of interferon lambda are provided herein.

In some embodiments, interferon lambda (e.g., an interferon lambda 1 such as interferon lambda 1a) or an analog thereof is formulated and/or administered and/or modified as described in one of the following patent publications, incorporated by reference herein: U.S. Pat. Nos. 6,927,040, 7,038,032, 7,135,170, 7,157,559, and 8,980,245, US 2009/0326204, US2010/0222266, US2011/0172170, or US2012/0036590.

### III. Examples

The following examples are provided to illustrate, but not to limit, the claimed invention.

### Example 1. Clinical Study Protocol for Treating HDV Subjects with Pegylated Interferon Lambda

This example describes a Phase 2 clinical study protocol for evaluating the safety, tolerability, and pharmacodynamics of pegylated interferon lambda monotherapy in subjects with chronic HDV infection.

TABLE 1
Protocol synopsis

| | |
|---|---|
| Product | Pegylated interferon lambda-1a (PEG-IFN-λ) |
| Study phase | Phase 2 |
| Indication | Chronic hepatitis D viral infection |

| | |
|---|---|
| Objectives | To evaluate the safety and tolerability of treatment with 2 dose levels of |
| | Lambda over a 48-week treatment period |
| | To evaluate the proportion of subjects with undetectable HDV RNA 12 weeks |
| | after the end of treatment |
| | To evaluate the effect of treatment with 2 dose levels of Lambda on the |
| | following: |

HDV levels
ALT levels

Hepatitis B surface antigen (HbsAg) levels
To evaluate the effects of Lambda treatment on immunologic parameters
Liver histology parameters

| | |
|---|---|
| Study design | Randomized, open-label study of Lambda 120 or 180 μg subcutaneous (SC) |
| | injection weekly for 48 weeks in subjects with chronic HDV infection. Subjects will |
| | also take an anti-HBV nucleos(t)ide analog (NUC) from baseline (Day 1) through |
| | the end of the study. Clinic visits at baseline (Day 1), Weeks 1, 4, and every 4 |
| | weeks until Week 48. PD/efficacy of Lambda will be assessed by measuring HDV |
| | and HBV viral loads, viral serologies. Safety and tolerability of Lambda will be |
| | assessed by AE monitoring, clinical laboratory tests, physical examinations, vital |
| | signs, body weight, and concomitant medications. All enrolled subjects will be |
| | followed for an additional 24 weeks off-treatment. All monthly follow-up visits will |
| | include evaluations of viral load (HDV and HBV), quantitative HbsAg (qHBsAg), and |
| | all of the safety measures listed above. |
| Study population and | Thirty-three subjects, 16-17 in each treatment group (120 or 180 μg daily) with |
| number of subjects | chronic HDV infection with detectable HDV RNA by quantitative polymerase chain |
| | reaction (qPCR) will be enrolled. |
| | Subjects who discontinue the study before Week 12 for reasons other than an |
| | adverse event (AE) may be replaced on approval of the sponsor. |
| Test product, dose, and | Pegylated interferon lambda-1a (PEG-IFN-λ) (Lambda), 120 or 180 μg, weekly SC |
| method of administration | administration |
| Duration of treatment | 48 weeks |
| Evaluation | The endpoints include the following: |

Proportion of subjects with undetectable HDV RNA 12 weeks after EOT (SVR-12)
Change from baseline in HDV viral load at Week 48 (EOT)

Additional PD/efficacy endpoints include:

Proportion of subjects with undetectable HDV RNA 12 weeks after EOT (SVR-24)
Change from baseline in HDV viral load
Change from baseline in HBV viral load
Change from baseline in HBsAg levels
Clearance of HBsAg
Change from baseline in Fibroscan
Liver biopsy improvement from baseline to Week 72 (EOFU)

Safety endpoints include:

Treatment-emergent AEs and SAEs
Treatment-emergent treatment-related AEs and SAEs
AEs leading to early discontinuation of study treatment
AEs leading to dose reduction
Treatment-emergent changes in clinical laboratory findings
Treatment-emergent changes in vital signs
Treatment-emergent changes in ECG findings
Treatment-emergent changes in physical examination results
Usage of concomitant medications during the study

At least one subject from the cohort of subjects who receive at least 80% of the total study drug dose throughout the entire 48-week treatment period and for whom HDV viral load data are available for the Day 1 (baseline) and end-of-treatment (Week 48) study visits shows improvement in one or more endpoints as described in the protocol. In some embodiments, a subject exhibits a reduction in HDV viral load at end-of-treatment as compared to baseline. In some embodiments, a subject exhibits a reduction in HBV viral load at end-of-treatment as compared to baseline. In some embodiments, interferon lambda therapy reduces HDV viral substantially, such as to an undetectable level as measured 12 weeks after end-of-treatment. In some embodiments, a subject exhibits a reduction in the level of HBsAg at end-of-treatment as compared to baseline. In some embodiments, a subject exhibits improved clearance of HBsAg antigen. In some embodiments, a subject exhibits a reduction in alanine aminotransferase (ALT) level. In some embodiments, a subject having a serum ALT that is above the upper limit of normal (ULN) prior to the onset of treatment exhibits an improvement in serum ALT level to a level that is within the ULN.

### Example 2. Pegylated Interferon Lambda Monotherapy for Treating HDV

This example describes interim data at 24 weeks from a phase 2 pegylated interferon lambda-1a (LIMT) clinical study conducted according to the protocol described in Example 1.

Background: Globally 15-20 million people are coinfected with hepatitis delta (HDV) and hepatitis B (HBV) viruses. Interferon (IFN) or pegylated (PEG) IFN-alfa have been tested in subjects with chronic HDV. Up to 25% of subjects may become HDV PCR-negative, but most relapse after therapy is discontinued and the tolerability profile is unsatisfactory. PEG IFN lambda-1a (“Lambda”) is a Type III IFN. Based on Lambda's more limited receptor distribution, it is postulated that Lambda could induce HDV responses, but with fewer side effects than IFN-alfa. LIMT HDV is the first study of Lambda in subjects with chronic HDV infection, including cirrhotics.

Randomized open-label study of Lambda 120 or 180 μg subcutaneous injections administered weekly for 48 weeks in subjects with chronic HDV. Major inclusion criteria were: positive HDV RNA by qPCR, elevated ALT<10×ULN, compensated liver disease and platelets ≥90,000 cells/μL. HDV RNA (Robogene 2.0, LLOQ 14 IU/mL), ALT, bilirubin and other parameters were assessed at each visit. Tenofovir or entecavir were started at baseline (BL) and continued through the end of the study. The primary endpoint was change from baseline in HDV viral load.

A total of 33 subjects were enrolled. Subcutaneous injections of Lambda (120 μg or 180 μg) were administered weekly for 48 weeks in subjects with chronic HDV. 16 subjects were randomized to Lambda 180 ag/week and 17 subjects were randomized to 120 g/week. The median characteristic values at baseline for the subjects are shown in Table 2 below. Due to increased frequency of liver-related SAEs at the Karachi, Pakistan site (observed in 7/15 [46.7%] subjects), the 6 subjects randomized at 180 mcg/week at the Karachi site were all reduced to 120 mcg/week (prior to the first dose). As a result of these changes in dosing regimens, for the efficacy and safety evaluation presented herein, subjects are categorized by their starting Lambda dose rather than randomization treatment group: 14/33 subjects at Lambda 180 mcg (“180 dose”) and 19/33 subjects at Lambda 120 mcg (“120 dose”).

TABLE 2
Baseline Characteristics

| Median Characteristic Values | Values | |
|---|---|---|
| N | 33 | |
| Age, years (range) | 36 | (20, 63) |
| Male, n (%) | 22 | (66.7%) |
| Race, n (%) | | |
| White | 13 | (39.4%) |
| Black | 1 | (3.0%) |
| Pacific Islander | 4 | (12.1%) |
| Other | 15 | (45.5%) |
| BMI, kg/m² (range | 24.7 | (14.0, 37.1) |
| HDV-RNA, log₁₀ IU/mL (range) | 4.4 | (2.4, 5.9) |
| ALT, U/mL (range)¹ | 84 | (35, 364) |
| Platelets, ×10⁹/L (range) | 170 | (95, 281) |
| Albumin, g/dL (range) | 4.4 | (3.7, 5.2) |
| INR | 1.2 | (1.0, 1.5) |
| Bilirubin, mg/dL (range)² | 0.5 | (0.2, 1.2) |

¹Normal range for ALT = 10-35 U/ml (female); 10-50 U/ml (male)
²Normal range for bilirubin = 0-1.2 mg/dL

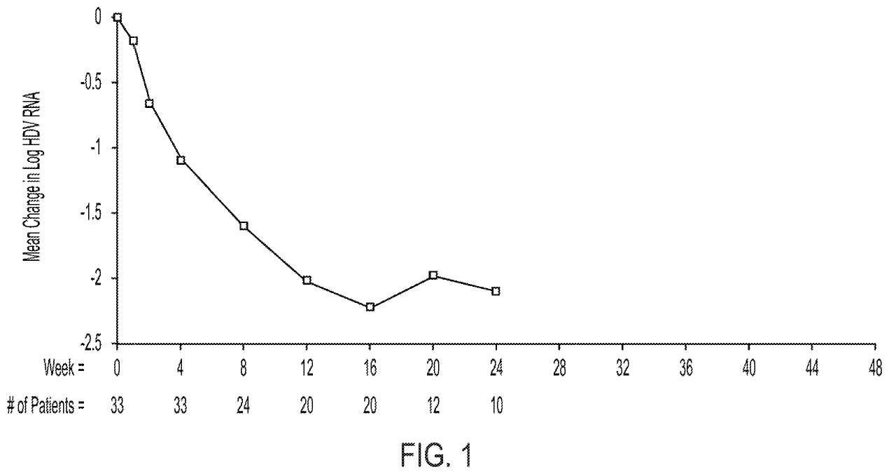

At the time of the interim analysis, all subjects had reached at least week 4 of therapy, and some subjects had reached week 8, 12, or 24 of therapy. FIG. 1 shows that subjects treated with interferon lambda demonstrated a rapid decline in HDV RNA. As shown in Table 3 below, for subjects who had reached week 24 of therapy, 50% achieved ≥2.0 decline in HDV RNA. 40% of the subjects were HDV PCR-negative. Mild to moderate headache, pyrexia, fatigue, and myalgia were the most commonly reported AEs. Per protocol dose reductions (12%), interruptions (12%), and treatment discontinuations (15%) were mainly due to hepatic AEs (ALT flares and/or hyperbilirubinemia). ALT flares and liver function abnormalities were generally correlated with HDV viral load decline. No cases of clinical decompensation were observed.

TABLE 3
Anti-HDV Activity

| Week | N | ≥2 log₁₀ decline | | PCR-negative | |
|---|---|---|---|---|---|
| 4 | 33 | 7 | (21.2%) | 3 | (9.1%) |
| 8 | 32 | 12 | (36.4%) | 5 | (15.6%) |
| 12 | 23 | 9 | (39.1%) | 4 | (17.4%) |
| 24 | 10 | 5 | (50.0%) | 4 | (40.0%) |

This interim analysis indicates that weekly Lambda-120 μg or 180 μg—has antiviral activity against HDV, with some subjects already becoming PCR-negative by Week 8 of therapy. Lambda demonstrates comparable anti-HDV activity to historical PEG-alfa at 24 weeks of treatment. Additionally, Lambda therapy was well-tolerated in the majority of subjects.

### Example 3. Pegylated Interferon Lambda Monotherapy for Treating HDV

This example describes end-of-treatment data at 48 weeks from a phase 2 pegylated interferon lambda-1a (LIMT) clinical study described in Example 1 and Example 2. Methods and baseline characteristics for the subjects are described in Example 2 and Table 2 above.

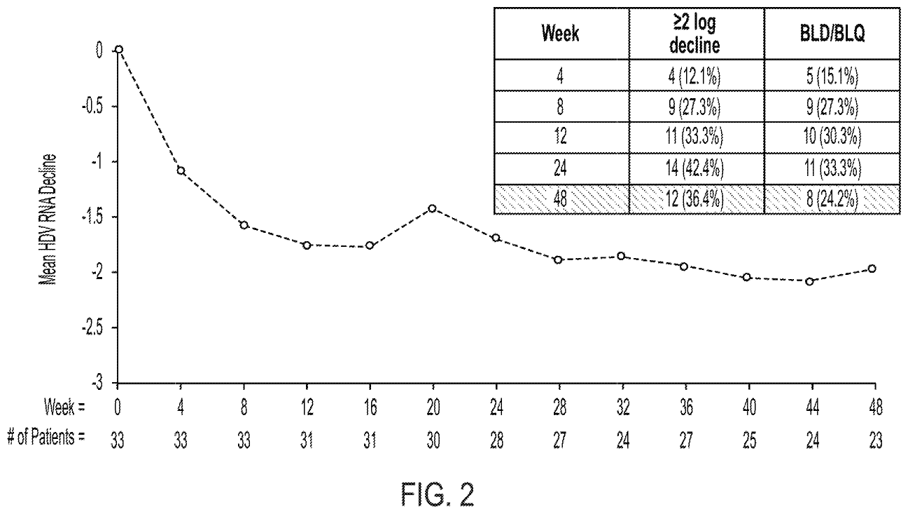

23 of the 33 subjects reached Week 48 (end of treatment), and 10 subjects discontinued treatment. As shown in FIG. 2, subjects treated with interferon lambda demonstrated a rapid decline in HDV RNA. Of the 33 subjects, 20 (60.6%) were responders at Week 48, defined as ≥2 log₁₀ decline in HDV RNA or HDV RNA below the limit of quantitation or below the limit of detection (BLQ/BLD). For BLQ, the limit of quantitation was 14 IU/mL. For BLD, the limit of detection was 8 IU/mL. HDV RNA data is also shown in Table 4 below. The anti-HDV activity of interferon lambda is comparable to historical data for pegylated interferon-alfa.

TABLE 4
End of Treatment (Week 48) Anti-HDV Activity

| Week | Mean VL Decline | ≥2 log₁₀ decline | | # BLQ/BLD* (%) | |
|---|---|---|---|---|---|
| 4 | −1.09 | 4 | (12.1%) | 5 | (15.1%) |
| 8 | −1.58 | 9 | (27.3%) | 9 | (27.3%) |
| 12 | −1.76 | 11 | (33.3%) | 10 | (30.3%) |
| 16 | −1.76 | 11 | (33.3%) | 13 | (39.4%) |
| 20 | −1.42 | 12 | (36.4%) | 11 | (33.3%) |
| 24 | −1.69 | 14 | (42.4%) | 11 | (33.3%) |
| 28 | −1.90 | 11 | (33.3%) | 9 | (27.3%) |
| 32 | −1.85 | 11 | (33.3%) | 8 | (24.2%) |
| 36 | −1.95 | 12 | (36.4%) | 9 | (27.3%) |
| 40 | −2.04 | 13 | (39.4%) | 10 | (30.3%) |
| 44 | −2.08 | 14 | (42.4%) | 9 | (27.3%) |
| 48 (End) | −1.96 | 12 | (36.4%) | 8 | (24.2%) |

*BLQ = below limit of quantitation (14 IU/mL); BLD = below limit of detection (8 IU/mL)

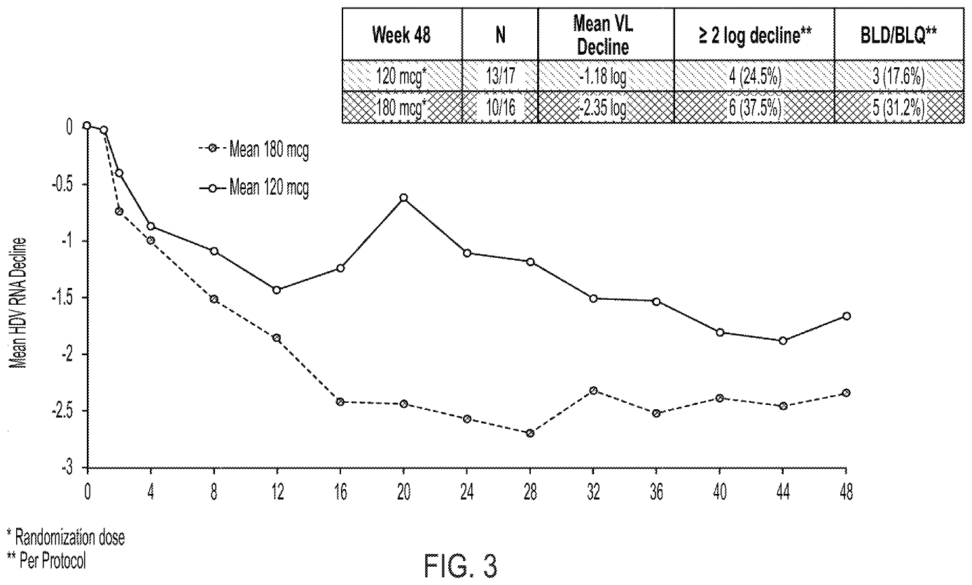

As shown in FIG. 3, it was found that subjects who were treated at the 180 mcg lambda dose demonstrated a higher response rate than subjects who were treated at the 120 mcg lambda dose, regardless of per protocol dose reductions. For subjects treated with 180 mcg interferon lambda, there was a mean decline of −2.35 log₁₀ HDV RNA at Week 48, as compared to −1.18 log₁₀ HDV RNA for subjects treated with 120 mcg interferon lambda. A greater number of subjects in the 180 mcg treatment group exhibited a ≥2 log₁₀ decline in HDV RNA (6 subjects, 37.5%) and had HDV RNA BLD/BLQ (5 subjects, 31.2%). In summary, interferon lambda treatment is well tolerated and demonstrates comparable anti-HDV activity to pegylated interferon alfa.

### Example 4. End of Study Results from LIMT HDV Study: 36% Durable Virologic Response at 24 Weeks Post-Treatment with Pegylated Interferon Lambda Monotherapy in Subjects with Chronic Hepatitis Delta Virus Infection

Background: Hepatitis Delta Virus (HDV) infection leads to the most aggressive form of human viral hepatitis. There is no approved therapy. Worldwide prevalence of HDV infection is 15-20 million. PEG IFN-lambda-1a (Lambda) has previously demonstrated a good tolerability profile in >3000 HBV and HCV subjects, with fewer cytopenias, flu-like and psychiatric symptoms compared to PEG IFN-alfa (Alfa). This study, LIMT, was designed to evaluate safety and efficacy of interferon lambda monotherapy (“Lambda”) in subjects with HDV.

This was a randomized, open-label study of Lambda 120 or 180 μg, weekly SC injections for 48 weeks follow by 24 weeks post-treatment in subjects with chronic HDV.

Inclusion criteria included positive HDV RNA by qPCR (Robogene© 2.0, BLQ 14 IU/mL), ALT<10×ULN, and compensated liver disease. Tenofovir or entecavir were started at baseline (BL).

In this study, 33 subjects were randomized to Lambda 180 μg (N=14) or 120 μg (N=19). BL mean values: HDV RNA 4.1 log₁₀ IU/mL (SD±1.4); ALT 106 IU/L (35-364) and bilirubin 0.5 mg/dL (0.2-1.2). See Table 5 below.

TABLE 5
48-Week Treatment and 24-Week Post-Treatment; HDV RNA Response (BLQ) by
Viral Load at Baseline (Lambda 180 mcg Group); and HDV RNA Response (Undetectable) by
Viral Load at Baseline (Lambda 180 mcg Group)

| | | | 48 Week | | | | | | |
|---|---|---|---|---|---|---|---|---|---|
| | | | On-Treatment | | | | 24 Week | | |
| | | | Mean | | | | Post-Treatment | | |
| | | | Week 48 | ≥2 Log | | | ≥2 Log₁₀ | | |
| | | | Log₁₀ HDV | Decline | | | Decline | | |
| Dose | | N | RNA Decline | or BLQ | BLQ | Undetectable | or BLQ | BLQ | Undetectable |
| 180 μg | All | 14** | −2.3 | 9/14 | 5/14 | 4/14 | 7/14 | 5/14 | 4/14 |
| | | | | 64% | 36% | 29% | 50% | 36% | 29% |
| | | 11*** | | 9/11 | 5/11 | 4/11 | 7/11 | 5/11 | 4/11 |
| | | | | 82% | 45% | 36% | 64% | 45% | 36% |
| | | 8** | | 7/8 | 3/8 | 2/8 | 4/8 | 2/8 | 2/8 |
| | High | | | 88% | 38% | 25% | 50% | 25% | 25% |
| | Baseline | 7*** | | 7/7 | 3/7 | 2/7 | 4/7 | 2/7 | 2/7 |
| | Viral | | | 100% | 43% | 29% | 57% | 29% | 29% |
| | Load | | | | | | | | |
| | Low | 6** | | 2/6 | 2/6 | 2/6 | 3/6 | 3/6 | 2/6 |
| | Baseline | | | 33% | 33% | 33% | 50% | 50% | 33% |
| | Viral | 4*** | | 2/4 | 2/5 | 2/5 | 3/5 | 3/5 | 2/5 |
| | Load* | | | 50% | 40% | 40% | 60% | 60% | 40% |

*Low baseline viral load = HDV RNA ≤4 log₁₀ IU/mL
**All subjects in trial at that starting dose
***All subjects that completed the trial and did not discontinue dosing

At Week 48, 36%-45% (of subjects in the Lambda 180 mcg group had an HDV RNA level below the LLOQ (BLQ). The proportion of these subjects with an HDV RNA level BLQ was comparable among those with a high vs low viral load at baseline (38% [3/8] and 33% [2/6], respectively) (Table 5).

At Week 24 post-dosing, the BLQ rate remained the same as at EOT for the Lambda 180 mcg group (36-45%) and was overall comparable to results at EOT among those with a high versus low viral load at baseline (25% [2/8] and 50% [3/6]), respectively) (Table 5).

Following Lambda 180 mcg/week treatment, 29-36% (4/14 total subjects, 4/11 completed subjects) of subjects reported undetectable HDV RNA at Week 48 as well as at Week 24 post-dosing (Table 8), further demonstrating the durability of virologic response to Lambda.

ITT rates of durable virologic response (DVR=BLQ at 24 weeks post-treatment) for Lambda 180 μg (5 of 14, 36%) compare favorably to historic rates for undetectability with Alfa 180 μg (0%) (Wedemeyer, 2019). 50% DVR in low BL viral load (VL) subjects (≤4 log₁₀) have been demonstrated with Lambda 180 mcg QW.

Common on-treatment AEs included mild to moderate flu-like symptoms and elevated transaminase levels. Subjects previously treated with Alfa noted significantly fewer side effects on Lambda. Cases of jaundice and increased incidences of bilirubin elevations were observed in the Pakistani cohort. No subjects showed symptoms of decompensation, and all responded favorably to dose reduction or dose discontinuation. DILIsym® modeling of ALT and bilirubin dynamics indicate a transporter-based mechanism for the observed bilirubin elevations.

Lambda 180 μg had better antiviral activity with better tolerability, compared to historical data for Alfa 180 mcg. Durable BLQ virologic responses have been observed 24 weeks post-treatment with Lambda 180 mcg.

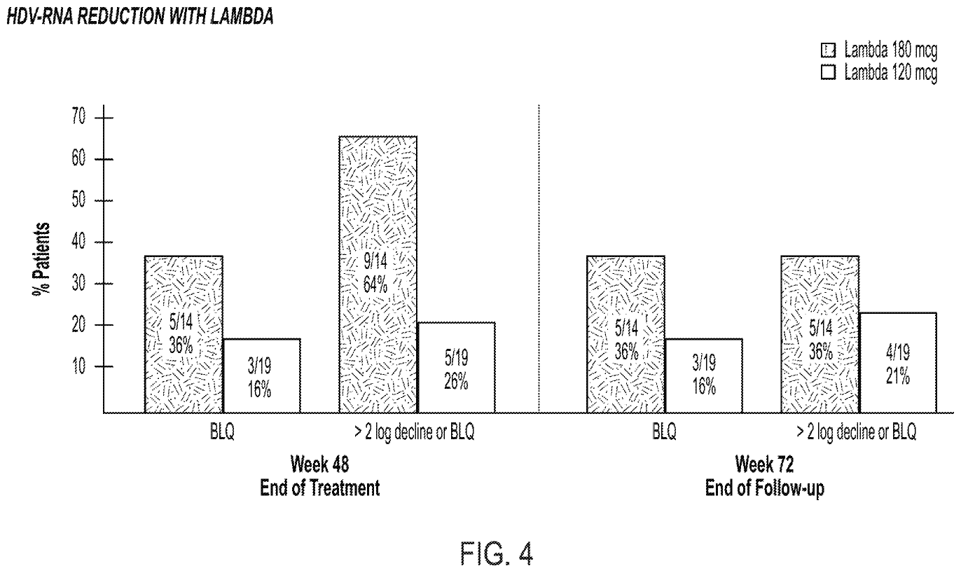

FIG. 4 demonstrates durable virologic responses (DVR) at 24 weeks post-treatment (Week 72) with both Lambda 180 and 120 mcg QW. Durable virologic responses (DVR) were defined as 24 week post-treatment responses of HDV RNA BLQ. It is thought that a 2 log₁₀ decline in HDV RNA may be a clinically meaningful reduction in HDV viral load, which could lead to improved survival (Farci et al 2004). PEG IFN alfa has demonstrated 23% undetectable HDV RNA at Week 48 with 0% of patients remaining undetectable at 24 weeks post-treatment.

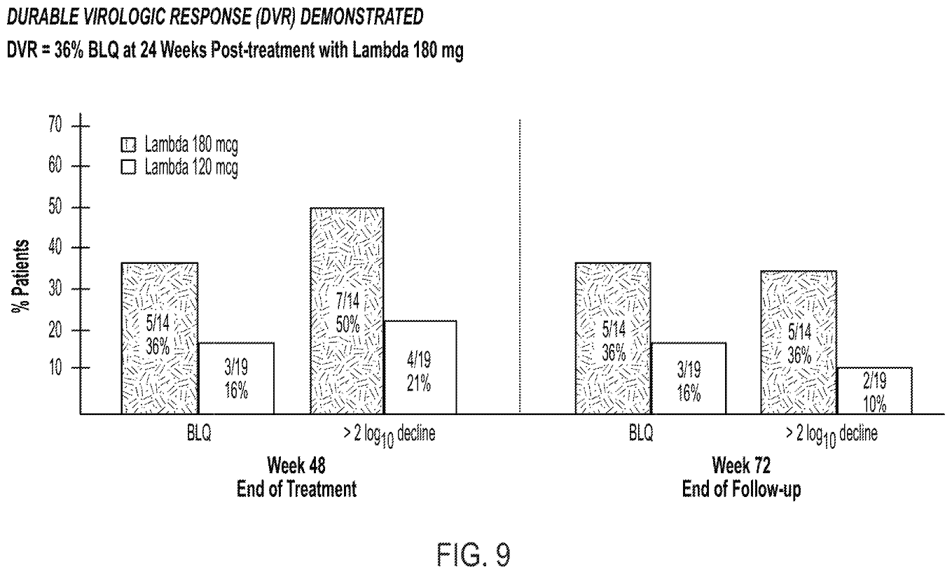

Surprisingly, what is shown here is that treatment with Lambda has an improved rate of between about a 36-45% durable response, which is better than the about 25% response obtainable with alpha. FIG. 9 and Table 6 further demonstrates that 36-45% of subjects treated with lambda who achieved BLQ after 48 weeks of treatment maintain HDV RNA BLQ at 24 weeks post treatment.

The subjects in the Lambda 180 mcg/week group, 50-64% at week 48 demonstrated >2 log₁₀ decline from baseline in HDV RNA. At Week 24 post last dose, 36-45% demonstrated >2 log₁₀ decline from baseline in HDV RNA (FIG. 9 and Table 6), which is clinically meaningful.

The subjects treated with Lambda 120 mcg/week, response rates at EOT and Week 24 post last dose were lower compared to response rates seen in the 180 mcg/week group. However, response rates at EOT were maintained at off-treatment follow-up as well. At Week 72, 16-21% (3/19) of subjects in the 120 mcg/week group demonstrated an HDV RNA BLQ and 11-14% (2/19) demonstrated >2 log₁₀ decline in HDV RNA from baseline (FIG. 9 and Table 6).

At Week 48, 14-18% and 11-14% of subjects treated with the Lambda 180 mcg/week and 120 mcg/week dose, respectively, displayed ALT normalization. At Week 24 post last dose, those numbers improved to 36-45% and 26-36%, respectively.

The majority of per protocol dose reductions (26-36% of subjects), interruptions (3-15% of subjects), and treatment discontinuations (21-26% subjects) the majority were related to hepatobiliary laboratory abnormalities (ALT, AST, GGT, and/or bilirubin increases). Of the 8 (24.2%) subjects who discontinued study drug treatment, 5 (62.5%) were from the Karachi, Pakistan site, which could be attributed to potential pharmacogenomic and/or environmental factors. No cases of clinical decompensation were observed. The percent of subjects being administered the 180 mcg/week dose have one or more of the following: dose reductions (about 12-35%), interruptions (about 7-15%), and treatment discontinuations (about 15-21%).

The percent of subjects being administered the 120 mcg/week dose have one or more of the following: dose reductions (about 12-35%), interruptions (about 7-15%), and treatment discontinuations (about 15-21%).

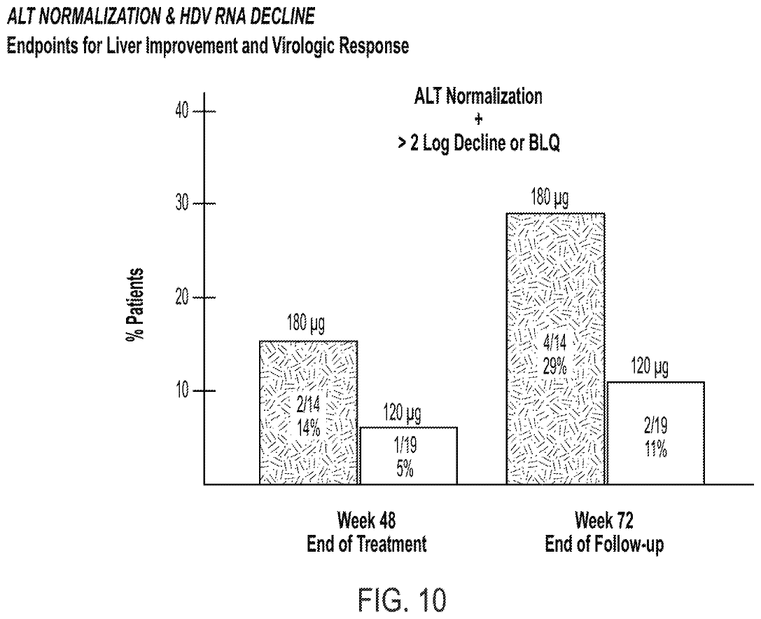

FIG. 10 and Table 6 demonstrates that subjects do achieve the composite endpoint of ALT normalization and a ≥2 log₁₀ decline at 24 weeks post-treatment which is also clinically meaningful. In addition, the proportion of subjects achieving the composite endpoint increases even after treatment had stopped, demonstrating a surprising finding of the benefit of treatment for HDV with Lambda. When combining ALT normalization with HDV RNA response, defined by either a viral load decline from baseline of ≥2 log₁₀ or an HDV RNA BLQ, in the Lambda 180 mcg/week group, 14-18% of subjects at Week 48 and 29-36% of subjects at Week 72 achieved the composite response. For the Lambda 120 mcg/week group, the composite response rates at Weeks 48 and 72 were 5-7% and 11-14%, respectively. In some subjects, there occurred an ALT flare from a baseline measurement (e.g., a transient increase), wherein a flare is 4× of one or more of: a baseline, from an end of treatment measurement, or from the upper limit of normal. In this study, about 24-32% of subjects had an ALT flare from the baseline measurement. As measured from the end of treatment, 12-16% of subjects flare. Of all subjects that experienced a flare, either as measured from a baseline or measured from end of treatment at 48 weeks, 44-92% subjects experienced ALT normalization after the flare. In some embodiments, the transient ALT increases are between about 300-1100% above the previous level or a baseline.

TABLE 6
Durable Virologic Response and ALT Normalization and HDV RNA Decline

| | Week 48 | | | | Week 72 | | | |
|---|---|---|---|---|---|---|---|---|
| | | | | ALT | | | | ALT |
| | | | | Normalization + | | | | Normalization + |
| Dose | | ≥2 log₁₀ | ALT | ≥2 log10 | | ≥2 log₁₀ | ALT | ≥2 log₁₀ |
| (mcg/week)* | BLQ | decline | Normalization | decline | BLQ | decline | Normalization | decline |
| 120** | 3/19 | 4/19 | 2/19 | 1/19 | 3/19 | 2/19 | 5/19 | 2/19 |
| | (16%) | (21%) | (11%) | (5%) | (16%) | (11%) | (26%) | (11%) |
| 120*** | 3/14 | 4/14 | 2/14 | 1/14 | 3/14 | 2/14 | 5/14 | 2/14 |
| | (21%) | (29%) | (14%) | (7%) | (21%) | (14%) | (36%) | (14%) |
| 180** | 5/14 | 7/14 | 2/14 | 2/14 | 5/14 | 5/14 | 5/14 | 4/14 |
| | (36%) | (50%) | (14%) | (14%) | (36%) | (36%) | (36%) | (29%) |
| 180*** | 5/11 | 7/11 | 2/11 | 2/11 | 5/11 | 5/11 | 5/11 | 4/11 |
| | (45%) | (64%) | (18%) | (18%) | (45%) | (45%) | (45%) | (36%) |

Starting dose. Due to increased frequency of liver-related SAEs at the Karachi, Pakistan site (observed in 7/15 [46.7%] subjects), the 6 subjects randomized at 180 mcg/week at the Karachi site were all reduced to 120 mcg/week (prior to the first dose when able). As a result of these changes in dosing regimens, for the efficacy and safety evaluation presented in this topline report, subjects are categorized by their starting Lambda dose rather than randomization treatment group: 14/33 subjects at Lambda 180 mcg and 19/33 subjects at Lambda 120 mcg.
*Starting dose
**All subjects in trial
***All subjects that completed the trial and did not discontinue dosing

In the Lambda 180 μg treatment group, response rates differed between subjects with high (>4 log₁₀) versus low (≤4 log₁₀) baseline viral load. At week 48, 38-43% and 33-40% of subjects with high versus low baseline viral loads respectively, reached HDV RNA levels BLQ. At week 72, the difference between these two groups became more prominent, with 50-60% of subjects in the low baseline viral load reaching BLQ versus 25-29% in the high baseline viral load meeting this endpoint (Table 6).

At week 48, 25-29% and 33-40% of subjects with high versus low baseline viral loads respectively, reached undetectable levels of HDV RNA. At week 72, there were difference between these two groups were consistent with the 48 week measure, with 33-40% of subjects in the low baseline viral load reaching BLQversus 25-29% in the high baseline viral load meeting this endpoint (Table 6).

Maintaining this response post-treatment may indicate a subjects' immune response gaining control over viral replication. Surprisingly, a higher percentage of patients with undetectable HDV RNA of 29% for Lambda 180 mcg QW at 24 weeks post-treatment is observed compared to that of pegylated interferon alfa-2a reported in a prior HDV study (0% in Myr203 study).

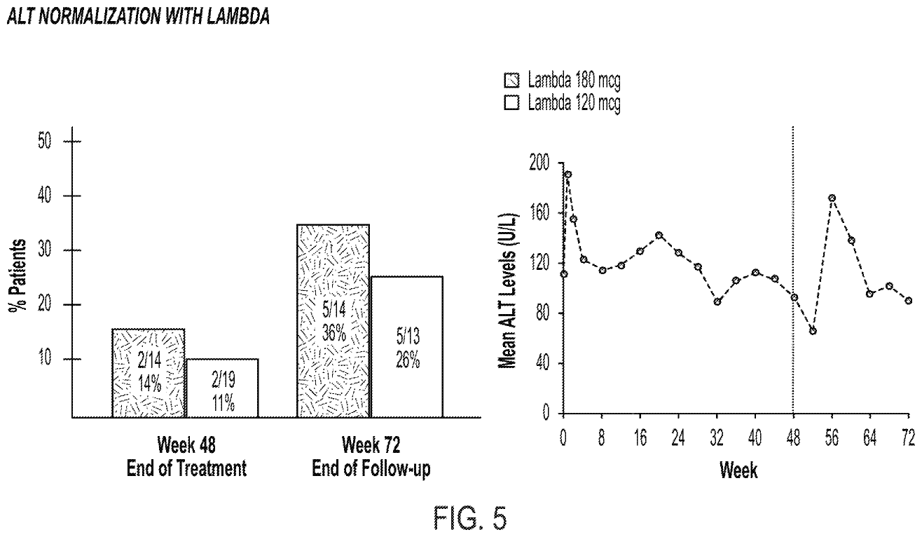

FIG. 5 demonstrates ALT normalization with Lambda. Alanine aminotransferase (ALT) normalization is a sign of improvement in liver health. ALT normalization was observed at end of treatment in 14% and 11% of subjects treated with Lambda 180 mcg QW and Lambda 120 mcg QW, respectively. ALT normalization continued to increase at 24 weeks post-treatment (Week 72).

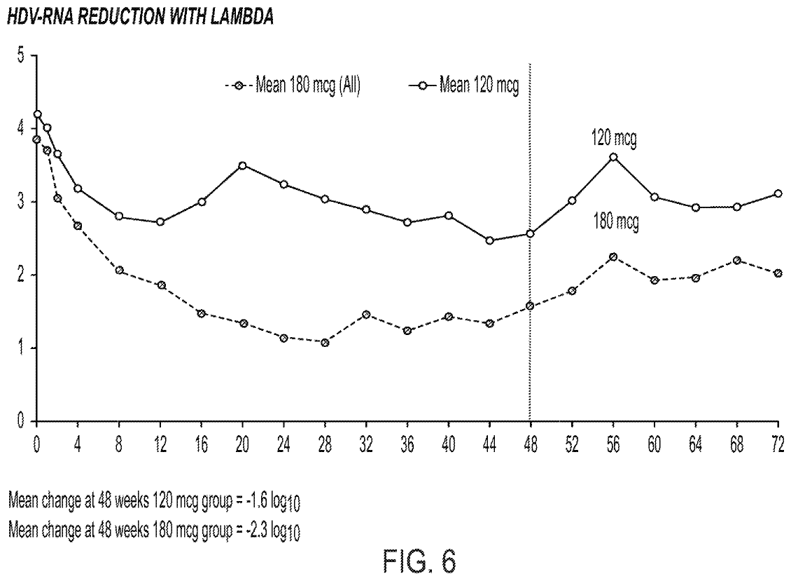

FIGS. 4 and 6 and Tables 7 and 8 demonstrate that Lambda 180 mcg QW results in larger HDV RNA decline compared to Lambda 120 mcg QW dose, despite dose reductions during treatment (180 mcg to 120 mcg or 120 mcg to 80 mcg).

TABLE 7

| | Week 48 | | Week 72 | |
|---|---|---|---|---|
| | | ≥2 Log₁₀ | | ≥2 Log₁₀ |
| Dose* | BLQ | Decline or BLQ | BLQ | Decline or BLQ |
| 120 | 3/19 | 5/19 | 3/19 | 4/19 |
| mcg** | 16% | 26% | 16% | 21% |
| 120 | 3/14 | 5/14 | 3/14 | 4/14 |
| mcg*** | 21% | 36% | 21$ | 29% |
| 180 | 5/14 | 9/14 | 5/14 | 5/14 |
| mcg** | 36% | 64% | 36% | 36% |
| 180 | 5/11 | 9/11 | 5/11 | 5/11 |
| mcg*** | 45% | 82% | 45% | 45% |

*Starting dose. Due to increased frequency of liver-related SAEs at the Karachi, Pakistan site (observed in 7/15 subjects), the 6 subjects randomized at 180 mcg/week at the Karachi site were all reduced to 120 mcg/week (prior to the first dose when able). As a result of these changes in dosing regimens, for the efficacy and safety evaluation presented in this topline report, subjects are categorized by their starting Lambda dose rather than randomization treatment group: 14/33 subjects at Lambda 180 mcg and 19/33 subjects at Lambda 120 mcg.
**All subjects in trial
***All subjects that completed the trial and did not discontinue dosing

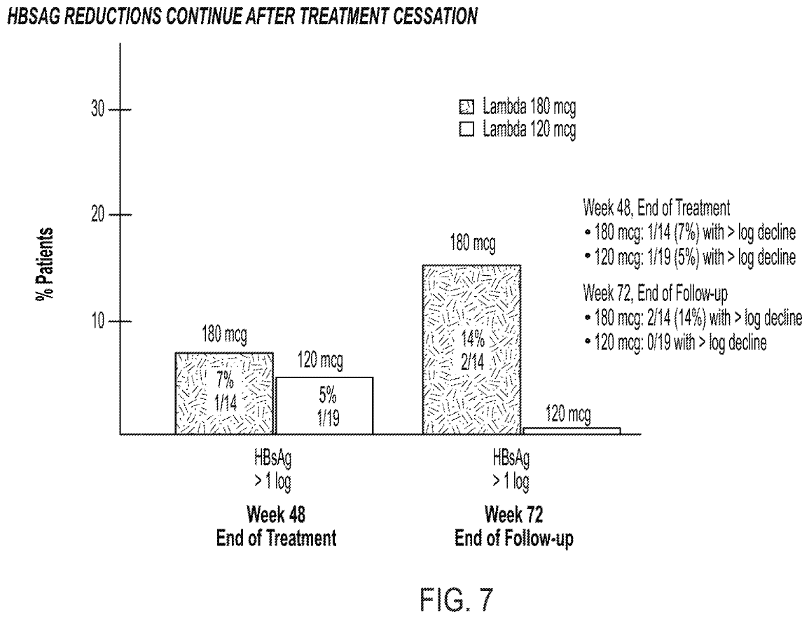

FIG. 7 demonstrates that Lambda treatment results in some subjects showing >1 log₁₀ decline in HBsAg. HBsAg levels continue to decline for some subjects post-treatment. Lower HBsAg levels may reflect HBeAg loss and HBV infectivity.

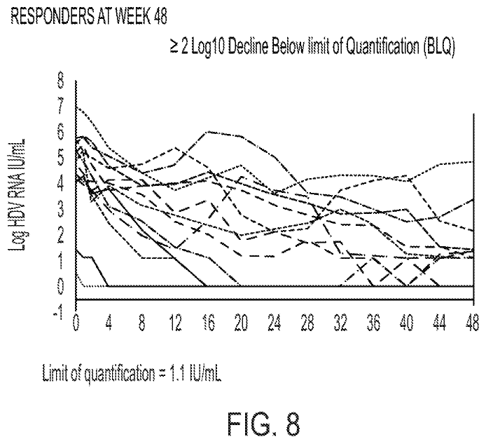

FIG. 8 demonstrate that HDV RNA of responders at Week 48 of treatment. Responders are defined as HDV RNA decline ≥2 Log₁₀ or Below Limit of Quantification (BLQ) at Week 48.

Table 8 shows the disposition of the subjects during the study. For example, 19 subjects were started at the 120 mcg dose and 14 subjects at the 180 mcg dose. However, 14 subjects remained in the study through Week 72 at the 120 mcg dose and 11 at the 180 mcg dose. Herein, some data is calculated with the enrolled and started subject number (modified intent to treat) (indicated at “N” in Table 8) and some data is calculated with reference to the “Remained in Study” (Per Protocol) number in Table 8

TABLE 8
Study Subject Dispositions

| | | Dose | Dose | Dose | Completed at | Remained | High Viral | Low Viral |
|---|---|---|---|---|---|---|---|---|
| Dose* | N | Reductions | Discontinuation | Interruption | Starting Dose | in Study | Load | Load |
| 120 mcg | 19 | 5 | 5 | 1 | 9 | 14 | — | — |
| 180 mcg | 14 | 5 | 3 | 2 | 6 | 11 | 8 | 6 |

*Starting dose (see above)

Table 9 shows that with Lambda treatment in this study, flu-like and psychiatric symptoms are predominantly grade 1. Cytopenias and thrombocytopenias (there were no thrombocytopenias) were much less frequent compared to historical pegylated interferon alfa use. There were milder and fewer flu-like and psychiatric symptoms with Lambda in the study and there were no thrombocytopenia events. There were elevated bilirubin and ALT levels normalized upon dose reduction or treatment discontinuation.

TABLE 9
Symptoms with Lambda are less severity (lower
grade) compared to pegylated interferon alfa

| AE | % Subjects Reporting | |
|---|---|---|
| Flu-like symptoms^{a} | 97% | (32/33) |
| Psychiatric symptoms^{b} | 3% | (1/33) |
| Cytopenia | 15% | (5/33) |
| Thrombocytopenia | 0% | (0/33) |
| Elevated bilirubin^{c,d} | 30% | (10/33) |
| Elevated ALT^{d} | 27% | (9/33) |

^{a}Flu-like symptoms: pyrexia, cough, sore throat, runny/stuffed nose, myalgia/arthralgia, headache, asthenia, vomiting, diarrhea
^{b}Psychiatric symptoms: depression, irritability, insomnia
^{c}11 of 18 events experienced by 4 subjects in Pakistan site
^{d}Labs normalized upon dose reduction or treatment discontinuation

TABLE 10
Adverse Events

| | | Number of Subjects Experiencing | | | |
|---|---|---|---|---|---|
| | | Grade of AE (N = 33) | | | |
| Classification | Adverse Event | Gr 1 | Gr 2 | Gr 3 | Gr 4 |
| Constitutional | fatigue, asthenia | 10 | 2 | — | — |
| Flu-like | pyrexia, chills, chest pain, flu-like | 5 | 1 | — | — |
| Neurological | dizziness, headache | 13 | 6 | 2 | — |
| Musculoskeletal | arthralgia, myalgia, back pain, | 15 | 5 | 1 | — |
| | musculoskeletal pain | | | | |
| Psychiatric | depression, irritability, insomnia | 1 | — | — | — |
| Hematological | neutrophil count decreased | 1 | 1 | — | 1 |
| Lab | bilirubin/ALT/AST/GGT increase | 2 | 1 | 11 | 1 |
| Abnormalities | | | | | |

Table 10 shows that in this study, there were milder flu-like and psychiatric symptoms with Lambda as compared with previous studies with alfa. There were no thrombocytopenia events, no use of hematopoetic growth factors, and elevated bilirubin and ALT levels normalized upon dose reduction or treatment discontinuation. In a head to head study of Lambda vs Alfa in a Phase 2 study in 176 HBV-infected subjects (LIRA-B), the overall frequency of events of clinical interest (constitutional symptoms, neurologic events, flu-like symptoms, musculoskeletal symptoms, and psychiatric events) was higher in the alfa group (72.3%) than in the Lambda 180-μg group (50.0%).

Clinical laboratory test abnormalities were consistent with the known safety profiles of Lambda and alfa, with increased frequencies of ALT, AST, and bilirubin (Grade 1 to 4 and Grade 3/4) in the Lambda 180-μg group compared with the alfa group; and increased frequencies of cytopenias, particularly leukopenia, neutropenia, and thrombocytopenia (Grade 1 to 4) in the alfa group compared with the Lambda 180-μg group. Milder and fewer flu-like and psychiatric symptoms were observed in Lambda when compared to other interferons such as Alfa. No events of thrombocytopenia were reported, and no hematopoetic growth factors were used. For events of elevated bilirubin, ALT, and/or AST levels, all occurrences normalized following dose reduction or treatment discontinuation.

Adverse events reported by subjects in the Lambda 180 mcg/week group were more severe for constitutional (fatigue, asthenia) and neurological (dizziness, headache) AEs but less severe for lab abnormalities (increased bilirubin, ALT, AST, GGT, or INR; DILI; decreased blood albumin; abnormal LFT) when compared with the Lambda 120 mcg/week group. See Tables 11 and 12.

TABLE 11
Treatment-emergent Adverse Events of Special Interest, by Maximum Severity
and Classification (Combined Lambda Dose Groups [180 mcg and 120 mcg])

| | | Number of Subjects Experiencing AE, | | | | | |
|---|---|---|---|---|---|---|---|
| | | by MaximumSeverity (N = 33) | | | | | |
| Classification | Adverse Event | G1 | | G2 | | G3 | G4 |
| Constitutional | fatigue, asthenia | 10 | (30.3) | 3 | (9.1) | — | — |
| Flu-like¹ | influenza-like | 5 | (15.2) | 1 | (3.0) | — | — |
| Neurological | dizziness, headache | 13 | (39.4) | 6 | (18.2) | 2 (6.1) | — |
| Musculoskeletal | arthralgia, myalgia, back pain, | 15 | (45.5) | 5 | (15.2) | 1 (3.0) | — |
| | musculoskeletal pain | | | | | | |
| Psychiatric | depression, irritability, insomnia | 1 | (3.0) | — | | — | — |
| Hematological | neutrophil count decreased | 1 | (3.0) | 1 | (3.0) | — | 1 (3.0) |
| Lab Abnormalities | increased bilirubin, ALT, AST, | 2 | (6.1) | 1 | (3.0) | 11 (33.3) | 1 (3.0) |
| | GGT, or INR; DILI; decreased | | | | | | |
| | blood albumin; abnormal LFT | | | | | | |

Abbreviations: AE = adverse event; ALT = alanine aminotransferase; AST = aspartate aminotransferase; DILI = drug-induced liver injury; GGT = gamma-glutamyl transferase; INR = international normalized ratio; LFT = liver function test.

TABLE 12
Treatment-emergent Adverse Events of Special Interest, by Maximum
Severity and Classification (Lambda 180 mcg Dose Group)

| | | Number of Subjects Experiencing AE, | | | | | | |
|---|---|---|---|---|---|---|---|---|
| | | by Maximum Severity (N = 14) | | | | | | |
| Classification | Adverse Event | G1 | | G2 | | G3 | | G4 |
| Constitutional | fatigue, asthenia | 6 | (42.9) | 3 | (21.4) | — | | — |
| Flu-like¹ | influenza-like | 1 | (7.1) | — | | — | | — |
| Neurological | dizziness, headache | 5 | (35.7) | 6 | (42.9) | 1 | (7.1) | — |
| Musculoskeletal | arthralgia, myalgia, back pain, | 7 | (50.0) | 2 | (14.3) | 1 | (7.1) | — |
| | musculoskeletal pain | | | | | | | |

| | | | | | | |
|---|---|---|---|---|---|---|
| Psychiatric | depression, irritability, insomnia | 1 | (7.1) | — | — | — |

| | | | | | | |
|---|---|---|---|---|---|---|
| Hematological | neutrophil count decreased | — | 1 | (7.1) | — | 1(7.1) |

| | | | | | | |
|---|---|---|---|---|---|---|
| Lab Abnormalities | increased bilirubin, ALT, AST, | — | — | 5 | (35.7) | 1(7.1) |
| | GGT, or INR; DILI; decreased | | | | | |
| | blood albumin; abnormal LFT | | | | | |

Abbreviations: AE = adverse event; ALT = alanine aminotransferase; AST = aspartate aminotransferase; DILI = drug-induced liver injury; GGT = gamma-glutamyl transferase; INR = international normalized ratio; LFT = liver function test.

In the study, there was a higher incidence of hyperbilirubinemia in Pakistan cohort. For example, the following parameters were different from the other cohorts, hyperbilirubinemia in 4/15 (27%) of Pakistani vs 2/18 (11%) of non-Pakistani cohort; jaundice observed in 3/15 (20%) of Pakistani subjects versus 0/18 (0%) of non-Pakistani subjects; incidence/severity in non-Pakistani cohort consistent with prior Lambda and Alfa data in HBV. That is subjects with bilirubin elevations did not experience signs or symptoms of decompensation and bilirubin levels were responsive to dose reduction/interruption and subjects exhibited normal hepatic function (PT) throughout periods of bilirubin elevation

In summary, of the 24 subjects who reached Week 48: 120 μg Group (N=14) had a mean HDV RNA decline=1.5 log₁₀; a ≥2 log₁₀ decline in 6 of 14 (42.9%); 180 μg Group (N=10) had a mean HDV RNA decline=2.4 log₁₀; and a ≥2 log₁₀ decline in 6 of 10 (60.0%). Lambda was well tolerated overall and increased incidences of clinical jaundice and bilirubin elevations were observed in the Pakistani cohort. This led to lower than expected rate of study completion (9 of 15, 60%) for Pakistan site, but none of the subjects with elevations in bilirubin showed symptoms of decompensation. All responded favorably to dose reduction or dose discontinuation.

The Lambda 180 mcg/week dose afforded greater efficacy than the Lambda 120 mcg/week dose, regardless of dose interruptions or reductions during treatment or dose discontinuations. About 35-45% of the subjects on the 180 mcg/week dose reduced to the 120 mg dose. About 7-9% of subjects on the 180 mcg/week dose, dose reduced twice.

Following 48 weeks of Lambda 180 mcg/week treatment, 36-45% of subjects achieved an HDV RNA level BLQ (29-36% undetectable) and 50% demonstrated a >2 log₁₀ decline from baseline in HDV RNA.

At 24-weeks post-dosing (e.g., after the final or last dose), this virologic response was maintained, with 36-45% of subjects evidencing an HDV RNA level BLQ (29% undetectable) with the Lambda 180 mcg/week treatment.

In comparison, a recent study using the same RoboGene assay (Kit 2.0) to measure HDV RNA for the efficacy of Myrcludex B in combination with PEG-IFNα in subjects with chronic HBV/HDV co-infection, reported 13% (2/15) of subjects with undetectable HDV RNA at Week 48 and 0% at 24-weeks post-dosing (72 weeks). (Wedemeyer et al, 2019), ILC 2019; GS-13

For subjects in the Lambda 180 mcg/week group, 36-45% reported ALT normalization at 24-weeks post-dosing, a trend observed in prior studies conducted with Lambda in subjects with HBV and HCV, presumably relating to Lambda's immune-modulatory effects on host effector cells, resulting in transient ALT increases during treatment followed by normalization post-treatment. In addition, 29-36% of subjects met the combined response criteria of ALT normalization and either HDV RNA BLQ or a decline from baseline of >2 log₁₀ at the 180 mcg/week dose.

At Week 48, 7 of 33 subjects had rebounded more than 1 log₁₀ increase from a previous measurement of the HDV RNA levels during the 48 week period. None of the 7 subjects that rebounded were found to meet BLQ at the end of 48 weeks. 21% of subjects who rebounded were not responders. 29% of the rebounding subjects were started at the 180 mcg dose.

Lambda PK has been characterized following single- and multiple-dose SC administration of Lambda in healthy subjects and patients with HCV (Table 12). The median time to maximum concentration (Tmax) ranged from 8.00 to 25.1 hours (range, 1-120 hours). Following single dose administration of Lambda 180 μg, the geometric mean maximum observed concentration (Cmax) (coefficient of variation [% CV]) values ranged from 1.06 (102) to 2.41 (177) ng/mL. Following multiple-dose administration, the geometric mean Cmax (% CV) was 1.54 (86.0) ng/mL, demonstrating modest accumulation. The area under the concentration-time curve from time zero extrapolated to infinite time (AUCinf) (% CV) following single-dose SC administration of Lambda 180 μg to healthy subjects and patients with HCV ranged from 116.9 (73.1) to 221 (59) ng×h/mL. In general, exposure values (area under the concentration-time curve [AUC] and Cmax) were approximately dose proportional in the 80 to 240 μg dose range. The mean (standard deviation [SD]) terminal elimination half-life (T1/2) ranged from 50.43 (20.47) to 74.0 (42.7) hours.

TABLE 12
Lambda Pharmacokinetic Parameters PK Parameters

| PK Parameter | Values |
|---|---|
| Tmax | 8.00 to 25.1 h Range (1-120 h) |
| Cmax | Single-dose: 1.06 (102) to 2.41 (177) ng/mL |
| AUCinf | Single-dose 180 [symbol] g to healthy subjects or patients with |
| T½ | |

Population PK modeling has demonstrated that body weight affects clearance, consistent with standard allometry; however, while body weight has a significant effect on clearance, the effect is small compared with the overall intersubject variability and, thus, does not warrant weight-based dosing. Preliminary results have shown that renal impairment increases exposure; Cmax and AUC were approximately 13% and 20% greater, respectively, in subjects with mild impairment and approximately 2-fold greater across the moderate renal dysfunction, severe renal dysfunction, and end-stage renal disease (ESRD) groups compared with subjects with normal renal function. Preliminary clinical results suggest that following single, 180-μg dose administration, Lambda is a mild inhibitor of CYP1A2, CYP2C9, and CYP3A4, and a moderate inhibitor of CYP2C19 and CYP2D6.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

What is claimed is:

1. A method of treating a hepatitis delta virus (HDV) infection in a human subject, the method comprising subcutaneously administering to the subject a therapeutically effective amount of pegylated interferon lambda-1a until one or more of a sustained reduction of HDV viral load is reached or a decrease in HDV RNA to undetectable levels.

2. The method of claim 1, wherein the pegylated interferon lambda-1a is administered for at least 12 weeks, or 24 weeks, or 36 weeks, or 48 weeks, or 54 weeks, or between 12 weeks and 96 weeks.

3. The method of claim 1, wherein the pegylated interferon lambda-1a is administered at a dose of 180 micrograms once a week (QW) or 90 micrograms twice per week; or 80 micrograms twice per week, or 180 micrograms per week.

4. The method of claim 1, wherein the pegylated interferon lambda-1a is administered at a dose of 120 micrograms QW, or 60 micrograms twice per week, or 70 micrograms twice per week, or 120 micrograms per week.

5. The method of claim 1, wherein the method comprises administering (i) 160-180 micrograms pegylated interferon lambda-1a per week for a first treatment period and then 150-70 micrograms per week for a second treatment period; or (ii) 180 micrograms per week for a first treatment period and then between 170-120 micrograms per week for a second treatment period, wherein the doses for each of (i) and (ii) may be divided into more than one dose per week.

6. The method of claim 1, wherein if the subject has an absolute neutrophil count (ANC) of between ≥to 500/mm³ and <750/mm³, or between ≥to 400/mm³ and <650/mm³, or between ≥to 400/mm³ and <850/mm³ at the end of the first treatment period, the subject will be administered pegylated interferon lambda-1a for the second treatment period.

7. The method of claim 5, wherein if the subject has an ANC of <500/mm³, dosing of the subject will be stopped until the ANC is >1000/mm³ and then dosing will resume for a second treatment period, or if the subject has an ANC of <400/mm³, dosing of the subject will interrupt dosing until the ANC is >750/mm³ and then dosing will resume for a second treatment period.

8. The method of claim 5, wherein if the subject has a platelet level of <50,000, the subject will be administered pegylated interferon lambda-1a for the second treatment period; or if a subject has a platelet level of <25,000; the subject will discontinue treatment.

9. The method of claim 5, wherein if the subject has an ALT (or AST)≥15-20×ULN and TBILI and/or INR<Grade 2, dosing of the subject will be interrupted dosing until the ALT/AST<10×ULN and then dosing will resume for a second treatment period; or if the subject has an ANC of ALT (or AST)≥15-20×ULN and TBILI and/or INR<Grade 2 for a second time, dosing of the subject will interrupt dosing until the ALT/AST<10×ULN and then dosing will resume for a second treatment period.

10. The method of claim 5, wherein if the subject experiences an adverse event≥Grade 3, dosing of the subject will be interrupted until the event resolves or is ≤a Grade 1 and then dosing will resume for a second treatment period.

11. The method of claim 10, wherein if the subject experiences a second adverse event of ≥Grade 3, dosing of the subject will be interrupted and then dosing will resume after the adverse event has resolved or improved by one Grade for a third treatment period.

12. The method of claim 1, wherein the method comprises administering the pegylated interferon lambda-1a 120 micrograms per week for a first treatment period and then 80 micrograms per week for a second treatment period; or 180-120 micrograms per week for a first treatment period and then 120-80 micrograms per week for a second treatment period, wherein the doses may be divided into more than one dose per week.

13. The method of claim 5 or 12, wherein the first treatment period is longer than the second treatment period, or the second treatment period is longer than the first treatment period, or first treatment period and the second treatment period are the same length of time.

14. The method of claim 5 or 12, wherein the first treatment period has a duration of at least 1 week, or at least 2 weeks, or at least 6 weeks, or at least 8 weeks.

15. The method of claim 5, wherein the first treatment period has a duration of 8-12 weeks.

16. The method of claim 5, wherein the method further comprises administering the pegylated interferon lambda-1a from between 80 micrograms-120 micrograms per week for a third treatment period.

17. The method of claim 1, wherein the method comprises administering the pegylated interferon lambda-1a at a first dose of 180 micrograms per for a first treatment period, at a second dose of 170-120 micrograms per week for a second treatment period, and at a third dose of 120-80 micrograms per week for a third treatment period.

18. The method of claim 17, wherein the first treatment period has a duration of from between 1-12, or 2-18, or 4-8, or 1-4, or 6-12 weeks.

19. The method of any of claims 1 to 12, wherein treatment results in a reduction of HDV viral load in the subject of at least 2.0 log HDV RNA IU/mL serum.

20. The method of any of claims 1 to 12, wherein treatment results in an HDV viral load that is below the level of detection.

21. The method of any of claims 1 to 12, wherein prior to the onset of treatment, the subject has a serum alanine aminotransferase (ALT) level that is above the upper limit of normal (ULN), and the course of treatment results in an improvement in serum ALT level in the subject to a level that is within the ULN.

22. The method of any of claims 1 to 12, wherein the method further comprises administering to the subject a nucleoside analog or nucleotide analog.

23. The method of claim 22, wherein the nucleoside analog or nucleotide analog is lamuvidine, adefovir, telbivudine, entecavir, or tenofovir.

24. The method of any of claims 1 to 12, wherein the subject has compensated liver disease with or without cirrhosis.

25. The method of claim 24, wherein the subject has compensated liver disease with cirrhosis.

26. The method of any of claims 1 to 12, wherein prior to treatment, the subject has a baseline viral load of up to about 10⁴ HDV RNA copies per mL serum or plasma.

27. The method of claim 1, 3 or 4, wherein a durable virologic response (DVR) is seen in the subject after administration.

28. The method of claim 27, wherein the DVR is about 16 to about 45%, or between about 36 to about 45%.

29. The method of claim 27, where the DVR is observed in the subject from about week 1 to about week 24 post treatment.

30. The method of claim 1, wherein administration of pegylated interferon lambda-1a causes milder and/or fewer flu-like and psychiatric symptoms compared with treatment with interferon alpha.

31. The method of claim 5 or 12, wherein an elevated bilirubin level and/or an ALT level identified in the subject normalize upon dose reduction.

32. The method of claim 31, wherein the subject has a chance of between about 11% to about 14% that ALT levels will normalize during treatment.

33. The method of any of claims 1 to 12, wherein treatment results in an HDV RNA decline in the subject of ≥2 Log₁₀.

34. The method of any of claims 1 to 12, wherein treatment results in a >1 log₁₀ decline in HBsAg in the subject.

35. The method of claim 34, wherein the subject's HBsAg levels continue to decline post-treatment.

36. The method of claim 3, wherein a mean decline of between −1.63 and −2.35 log₁₀ HDV RNA is observed in the subject at 48 weeks of treatment.

37. A method of treating a hepatitis delta virus (HDV) infection, comprising administering from about 80 to about 240 μg of pegylated interferon lambda-1a per week for at least four weeks, wherein between 1 day and 24 weeks post a last administration subjects have a durable virologic response (DVR).

38. The method of claim 37, wherein DVR comprises one or more of post-treatment responses of HDV RNA BLQ; a 2 log₁₀ or greater decline in HDV RNA; a HDV 2 log₁₀ or greater decline in viral load; ALT normalization; ALT normalization plus a >2 log₁₀ decline, or a clinically meaningful viral load decline.

39. The method of claim 38, wherein the viral load decline comprises between −1.09 and −2.08 or between −1.63 log₁₀ and −2.3 log₁₀.

40. The method of claim 38, wherein a subject has about a 12.1% to about 42.4% chance of the treatment resulting in the 2 log₁₀ or greater decline in HDV RNA.

41. The method of claim 38, wherein a subject has a 15.1% to about 39.4% chance of the treatment resulting in the HDV RNA being BLQ.

42. The method of claim 38, wherein a viral load decline between about −1.18 log₁₀ HDV RNA and about −2.35 log₁₀ HDV RNA is observed at 48 weeks of treatment.

43. The method of claim 38, wherein the subject has an increased chance of achieving ALT normalization and a >2 log₁₀ decline after a last administration than during administration.

44. The method of claim 43, wherein the last administration is between week 4 and week 48 of administration.

45. The method of claim 1, wherein a subject has a chance of about 36-45% of the treatment resulting in ALT normalization at 24-weeks post-dosing when administered 180 mcg/week.

46. The method of claim 45, wherein transient ALT increases occur during treatment followed by normalization post-treatment.

47. The method of claim 46, wherein the transient ALT increases are between about 300-1100% above the previous level or a baseline.

48. The method of claim 1, wherein a subject has a chance of between about 26-36% of reducing to a second dose during treatment; between about 5-9% chance of having a dose interruption, or between about 21-26% chance of discontinuing treatment.

49. The method of claim 48, wherein the reductions, interruptions, and discontinuations are primarily due to hepatic adverse events.

50. The method of claim 48, wherein the percent of subjects being administered the 180 mcg/week dose have one or more of the following: dose reductions (about 30-36%), interruptions (about 7-9%), and treatment discontinuations (about 21-24%).

51. The method of claim 48, wherein the percent of subjects being administered the 120 mcg/week dose have one or more of the following: dose reductions (about 26-30%), interruptions (about 5-9%), and treatment discontinuations (about 24-26%).

52. The method of claim 1, wherein 38-43% of subjects receiving a starting dose of 180 micrograms per week and after a last administration who had a high (>4 log₁₀) baseline viral load achieved HDV RNA levels BLQ at week 48.

53. The method of claim 1, wherein 25-29% subjects receiving a starting dose of 180 micrograms per week and after a last administration who had a high (>4 log₁₀) baseline viral load achieved HDV RNA levels BLQ at 24 weeks post treatment.

54. The method of claim 1, wherein 33-40% subjects receiving a starting dose of 180 micrograms per week and after a last administration who had a low (≤log₁₀) baseline viral load achieved HDV RNA levels BLQ at week 48.

55. The method of claim 1, wherein 50-60% subjects receiving a starting dose of 180 micrograms per week and after a last administration who had a low (≤log₁₀) baseline viral load achieved HDV RNA levels BLQ at 24 weeks post treatment.

56. The method of claim 1, wherein 25-29% subjects receiving a starting dose of 180 micrograms per week and after a last administration who had a high (>4 logs) baseline viral load achieved undetectable HDV RNA levels at week 48 and 24 weeks post treatment.

57. The method of claim 1, wherein 33-40% subjects receiving a starting dose of 180 micrograms per week and after a last administration who had a low (≤log₁₀) baseline viral load achieved undetectable HDV RNA levels at week 48 and 24 weeks post treatment.

58. The method of claim 1, wherein after a last dose one or more of: 16-21% subjects receiving a starting dose of 120 micrograms per week achieved HDV RNA levels BLQ; 21-29% subjects receiving a starting dose of 120 micrograms per week achieved >2 log₁₀ decline; 11-14% subjects receiving a starting dose of 120 micrograms per week achieved ALT normalization; 5-7% subjects receiving a starting dose of 120 micrograms per week achieved ALT Normalization+≥2 log₁₀ decline.

59. The method of claim 1, wherein 24 weeks after a last dose one or more of: 16-21% subjects receiving a starting dose of 180 micrograms per week achieved HDV RNA levels BLQ; 11-14% subjects receiving a starting dose of 180 micrograms per week achieved >2 log₁₀ decline; 26-36% subjects receiving a starting dose of 180 micrograms per week achieved ALT normalization; 11-14% subjects receiving a starting dose of 180 micrograms per week achieved ALT Normalization+≥2 log₁₀ decline.

60. The method of claim 1, wherein after a last dose one or more of: 36-45% subjects receiving a starting dose of 180 micrograms per week achieved HDV RNA levels BLQ; 50-64% subjects receiving a starting dose of 180 micrograms per week achieved ≥2 log₁₀ decline; 14-18% subjects receiving a starting dose of 180 micrograms per week achieved ALT normalization; 14-18% % subjects receiving a starting dose of 180 micrograms per week achieved ALT Normalization+≥2 log₁₀ decline.

61. The method of claim 1, wherein 24 weeks after a last dose one or more of: 36-45% subjects receiving a starting dose of 180 micrograms per week achieved HDV RNA levels BLQ; 36-45%% subjects receiving a starting dose of 180 micrograms per week achieved >2 log₁₀ decline; 36-45%% subjects receiving a starting dose of 180 micrograms per week achieved ALT normalization; 29-36% subjects receiving a starting dose of 180 micrograms per week achieved ALT Normalization+>2 log₁₀ decline.

62. The method of claim 1, wherein the subject has a baseline Child-Turcotte-Pugh score of 5-6 (class A), or 1-2, or 1-3, or 2-4, or 3-4, or 2-5, or 3-5 or 2-6.

63. The method of claim 1, wherein the subject has been diagnosed with hepatitis by one or more of: liver biopsy, liver function test, ultrasound, hepatic venous pressure gradient (HVPG) measurement, ALT level, other blood tests, or albumin level

64. The method of claim 63, wherein the serum alanine aminotransferase (ALT) level is determined within 24 weeks prior to treatment, at the initiation of treatment, within 24 months, 24 months-1 month, or within 12 months to 1 day prior to treatment.

65. The method of claim 1, wherein the subject's HDV titer rises from baseline to an elevated HDV titer prior to dropping below baseline during the course of treatment, wherein the subject's HDV level rises to more than 10%, more than 25%, more than 50%, more than 75%, more than 100%, more than 150%, or more than 200% of baseline, or between about 25-50% of a baseline, or from 25-100% of baseline, or from 50-200% of baseline.

66. The method of claim 65, wherein the rise in the subject's HDV titer occurs within 2 weeks after initiation of therapy.

67. The method of claim 66, wherein the subject's elevated HDV titer drops to below baseline within 2 weeks, or within 3 weeks, of initiation of therapy.

68. The method of claim 69, wherein the subject exhibits an improvement in one or more liver function parameters, wherein the improved liver function is an improvement in one or more serum markers. 70. The method of claim 68, wherein the one or more liver function parameters include one or more of serum albumin, bilirubin, alanine aminotransferase (ALT), aspartate aminotransferase (AST), prothrombin, alfa2-macroglobulin, apolipoprotein A1, haptoglobin, or gamma-glutamyl transpeptidase (GGT).

71. The method of claim 1, wherein the subject exhibits an improvement in liver fibrosis after treatment, or during treatment.

72. The method of claim 71, wherein the liver fibrosis is assessed by one or more of the following: biopsy with histological analysis, transient ultrasound elastography, or magnetic resonance elastography.

73. The method of claim 71, wherein the improvement in liver fibrosis is at least 5%, at least 10%, at least 20%, at least 30%, at least 40%, at least 50%, between 5-40%, between 10-50, 50-100% improved.

74. The method of claim 71, wherein improvement is measured by in functional parameters, wherein the functional parameters are one or more of an improvement in serum marker(s) or an improvement in liver fibrosis) as compared to a baseline.

75. The method of claim 74, wherein the baseline is one or more of at the onset of treatment, at another point during the course of treatment or as compared to a healthy subject.

76. The method of claim 1, wherein if a subject has a increase of greater than one log₁₀ in HDV RNA levels during treatment, as measured from a baseline, the subject discontinues treatment for one week, two, weeks, three, weeks, or until the subject has stabilized HDV viral load to a baseline level.

77. The method of claim 1, wherein after treatment begins, the subject has about a 24-32% chance of having an ALT flare from a baseline measurement; or 12-16% as measured from the end of treatment.

78. The method of claim 77, wherein a flare is a transient increase that is ≥4×: a baseline value, an end of treatment value, or from the upper limit of normal.

79. The method of claim 77, wherein 44-92% subjects experienced ALT normalization after the flare, wherein the flare is measured from a baseline or measured from end of treatment.

80. The method of claim 1, wherein prior to the onset of treatment, the subject has a serum alanine aminotransferase (ALT) level that is above the upper limit of normal (ULN), and the course of treatment results in an improvement in serum ALT level in the subject to a level that is within the ULN.

81. The method of claim 63, wherein the biopsy is within the 6 months before treatment; within the 18 months before treatment; within the 1 day to 24 months before treatment.


SEQUENCE LISTING (1 sequence)

SEQ ID NO: 1 - protein, 176 aa, Artificial Sequence
  Description of Artificial Sequence Synthetic polypeptide
MKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLSLRTSTHPEST